(19) United States
(12) Patent Application Publication
(10) Pub. No.: US 2012/0083518 A1
(43) Pub. Date: Apr. 5, 2012

(54) Cancer Treatment Targeting BRCA1-IRIS

(76) Inventor: WAEL M. ELSHAMY, MADISON, MS (US)

(21) Appl. No.: 13/236,292

(22) Filed: Sep. 19, 2011

Related U.S. Application Data
(60) Provisional application No. 61/383,995, filed on Sep. 17, 2010.

(51) Int. Cl. A61K 31/713; G01N 33/53; C12Q 1/68; A61P 35/00; A61P 35/04

(52) U.S. Cl. 514/44 A; 435/6.12; 435/7.1; 436/501

(57) ABSTRACT

Patients identified as candidates at risk of developing aggressive cancer, including metastatic cancer, are determined based on the expression of BRCA1-IRIS. In one advantageous form, the expression of BRCA1-IRIS is quantified based on BRCA1-IRIS present in tumor cells of patients. The amount of BRCA1-IRIS expression can be quantified in any conventionally known manner, including quantifying the amount of protein present in tumor cells or BRCA1-IRIS mRNA present in tumor cells of patients.

### CROSS REFERENCE TO RELATED APPLICATIONS

This application claims the benefit of provisional patent application No. 61/383,995, filed Sep. 17, 2010, herein incorporated by reference.

### FIELD OF THE INVENTION

The present invention relates to a therapeutic treatment of cancer, and in particular, a therapeutic treatment of cancer targeting BRCA1-IRIS.

### BACKGROUND OF THE INVENTION

Cancer is one leading cause of non-natural deaths around the world. One form of cancer, ovarian cancer, is the fourth leading cause of cancer deaths worldwide (1). Cisplatin and its analogues are first-line chemotherapeutic agents for the treatment of human ovarian cancer (2, 3). Cisplatin promotes its cytotoxicity by forming DNA-protein cross-links, DNA mono-adducts and intrastrand DNA cross-links all trigger apoptosis (4, 5). While the mechanisms involved in cisplatin resistance are not yet fully understood (6), decreased cellular detoxication (7), increased DNA repair (8), p53 mutations (9) as well as defects in intra- or extracellular survival/apoptotic pathways all have been implicated (10, 11).

Survivin is a bifunctional member of the inhibitor of apoptosis proteins (IAP) family (12) that participates in cell division control besides being a suppressor of apoptosis (13). Survivin inhibits apoptosis through binding to caspase3/7 and inhibits their functions (14). Altered survivin expression is common in many human cancers, such as ovarian, lung, colon, liver, prostate, and breast cancers (15-20) and reducing survivin level sensitizes tumor cells to anticancer drugs (21). Survivin expression is activated, in part, by AKT-dependent mechanism in many cell types, including ovarian cells (22).

AKT is a family of serine/threonine kinases activated in a phosphatidylinositol 3-kinase (PI3′K)-dependent manner by a variety of stimuli, including growth factors (23). AKT can suppress apoptosis by inhibiting pro-apoptotic proteins, such as BAD, caspase-9 and the transcription factor FKHRL1 (24, 25). AKT deregulation is also involved in the development of chemo-resistance (24, 25). AKT2 is amplified in many human ovarian cancer cell lines and primary ovarian carcinomas (26-38) and inhibition of PI3′K/AKT2 induces apoptosis in ovarian cancer cells that overexpresses AKT2 (29).

BRCA1-IRIS is a recently discovered, 1399 residue BRCA1 locus splice variant (30). Although it and the full-length product of this locus, the tumor suppressor BRCA1/p220 (31, 32), share 1365 residues, unlike BRCA1/p220, BRCA1-IRIS possesses oncogenic functions. BRCA1-IRIS induces DNA-replication by inhibiting Geminin negative function at DNA replication origins (30) and cell proliferation by up-regulating Cyclin D1 expression (33, 34). Furthermore, BRCA1-IRIS expression is high in multiple sporadic human breast and ovarian cancer cell lines, as well as known BRCA1^{mutant/-} cell lines, such as HCC1937 and SNU251 (35, 36).

Evasion of apoptosis plays a role in cancer development, drug resistance and reoccurrence. The BRCA1 locus product protein BRCA1-IRIS is over-expressed in several cisplatin resistant ovarian cancer cell lines, but its relationship to resistance was previously unknown.

### SUMMARY OF THE INVENTION

The present invention relates to identification of patients at risk of developing aggressive cancer based on the presence of BRCA1-IRIS expression. Overexpression of BRCA1-IRIS renders tumor cells resistant to current therapeutic methods of treatment, including chemotherapy and radiation. Overexpression of BRCA1-IRIS results in the tumor cells having anti-apoptosis characteristics. Further, the overexpression of BRCA1-IRIS is closely tied to the development of aggressive cancer, which includes resistance to current cancer therapies and metastatic cancer.

In accordance with one aspect of the present invention, a method provides for identifying patients at risk of developing aggressive cancer. The method includes selecting a patient to determine one's risk of developing aggressive cancer and acquiring a biological sample from a patient. The amount of IRIS expression is determined from the biological sample and the patient is identified as a risk candidate for developing aggressive cancer if the amount of BRCA1-IRIS expression exceeds a threshold amount of expression.

In various further specific embodiments, the method includes quantitative biological sample by biopsying a tumor from the patient and determining the amount of BRCA1-IRIS expression by determining the amount of BRCA1-IRIS protein present in the tumor and determining the amount of BRCA1-IRIS mRNA present in tumor cells.

The present invention, in another form, relates to a method for detecting expression of BRCA1-IRIS in a patient. The method includes selecting a patient to determine the amount of BRCA1-IRIS expression and acquiring a biological sample from the patient. The biological sample is contacted with a monoclonal antibody specific for an epitope of BRCA1-IRIS. The amount of BRCA1-IRIS present is quantified in the sample based on an amount of antibody bound to BRCA1-IRIS. In one specific form, the antibody recognizes the segment in intron 11 of BRCA1-IRIS.

The present invention in another form relates to a method for detecting expression of IRIS in a patient in which the amount of BRCA1-IRIS is determined by extracting mRNA from a biological sample of a patient and conducting PCR on mRNA in the biological sample using primers specific to a 5′ end and a 3′ end of a segment in BRCA1-IRIS. In one specific embodiment, the forward primer has sequence SEQ ID NO:1 and the reverse primer has sequence SEQ ID NO:2.

The present invention, in another form thereof, relates to a method for reducing the growth or proliferation of cancer cells. The method include administering a therapeutically effective amount of an inhibitor of BRCA1-IRIS to cancer cells to thereby reduce the growth or proliferation of the cancer cells. In various further embodiments, the method uses an inhibitor, which is siRNA, which binds to BRCA1-IRIS mRNA to thereby reduce the amount of BRCA1-IRIS protein produced by the cancer cells. In one advantageous form of the method, administering siRNA induces BRCA1-IRIS mRNA degradation when introduced to cancerous cells expressing normal or high levels of BRCA1-IRIS mRNA.

The present invention is directed to a present discovery that BRCA1-IRIS overexpression promotes the generation of breast, ovarian and other cancer stem cells that self-renew, evade/metasticize and are drug resistant and reoccur. Based on this discovery, an inhibitor of BRCA1-IRIS was developed which is directed to preventing or reducing the risk of the occurrence of breast, ovarian and other types of cancer mestatisize, drug resistance and reoccurrence.

Based on the present studies, a therapeutic method for treating or reducing the occurrence of cancer has been developed. The treatment is based on the discovery of a novel breast, ovarian and other cancer oncogene BRCA1-IRIS. The BRCA1-IRIS molecule is overproduced in breast, ovarian and other cancerous cells and leads to the development of aggressive and a deadly form of these cancers. The present therapeutic method targets BRCA1-IRIS with various agents such as chemotherapeutic drugs, antibodies or other agents targeting BRCA1-IRIS to treat aggressive/metastatic/drug resistant/recurrent breast, ovarian and other cancers.

The studies presented below in the detailed description show that in human ovarian surface epithelial (HOSE) cells overexpression of BRCA1-IRIS triggers expression of the anti-apoptotic protein survivin. Negative modulation of PI3K signaling or AKT silencing reduced survivin expression in this setting. Conversely, silencing BRCA1-IRIS in ovarian cancer cell lines derepressed PTEN expression along with the anti-apoptotic AKT targets FOXO1 and FOXO3a, suppressing survivin expression. Cisplatin (≦50 μM) exposure was sufficient to activate expression of the BRCA1-IRIS-AKT-survivin cascade in HOSE cells, whereas under similar conditions cisplatin failed to induce apoptosis in ovarian cancer cell lines expressing this regulatory cascade. Mechanistic investigations indicated that BRCA1-IRIS triggers survivin expression through a PI3K/AKT-dependent pathway involving NF-κB, but also through a PI3K/AKT-independent pathway involving PTEN, FOXO1, and FOXO3a. The present findings indicate how BRCA1-IRIS overexpression prevents chemotherapy-induced cell death by upregulating expression of survivin, and they highlight this regulatory cascade as a candidate focus to improve treatment of advanced drug-resistant ovarian cancers.

In accordance with the present disclosure, BRCA1-IRIS overexpression triggers survivin expression and ovarian cell survival, in part through triggering the expression and activity of AKT1, AKT2 and NF-κB and in part by repressing PTEN/FOXO1 and FOXO3a expression. This BRCA1-IRIS-AKT-survivin cascade was upregulated following treatment of ovarian surface epithelial cells (HOSE) with low cisplatin concentrations and promoted resistance to cisplatin-induced killing Furthermore, ovarian cancer cell lines endogenously overexpressing BRCA1-IRIS are more resistant to cisplatin. The present data provides evidence that BRCA1-IRIS overexpression is an important mediator of chemo-resistance in ovarian cancer cells. Accordingly, combinatorial-targeted inhibition of the BRCA1-IRIS-AKT-survivin pathway enhances the effectiveness of chemotherapy in the treatment of ovarian cancer.

### BRIEF DESCRIPTION OF THE FIGURES

FIGS. 1A and 1B relate to BRCA1-IRIS, AKTs and survivin expression in ovarian cancer cell lines wherein FIG. 1A is a real-time RT/PCR quantification of the relative levels of BRCA1-IRIS, BRCA1/p220, AKT1, 2, 3 and survivin mRNAs levels normalized to GAPDH mRNA levels in HOSE and ovarian cancer cell lines. Values represent means±SD of at least triplicates; and FIG. 1B is a Western blot analysis of several ovarian cancer cell lines for the indicated proteins.

FIGS. 2A-2H show ectopic expression of BRCA1-IRIS in HOSE cells promotes AKTs and survivin expression and phosphorylation where expression of indicated proteins in survivin wherein FIG. 2A is HA-AKT1, 2 and 3; FIG. 2B is BRCA1-IRIS, FIG. 2C is overexpressing HOSE cells. Expression of indicated proteins in SKOV3 cells AKT; FIG. 2D is BRCA1-IRIS; FIG. 2E is AKT and BRCA1-IRIS; FIG. 2F is surviving; FIG. 2G is silenced cells; and FIG. 2H is expression of indicated proteins in HOSE, HOSE/IRIS or HOSE/IRIS transfected with AKTs siRNA.

FIGS. 3A-3D show BRCA1-IRIS silencing in ovarian cancer cell lines reduces AKT and survivin expression and phosphorylation wherein FIG. 3A is expression of the indicated proteins in MACS and SKOV3 untreated, treated with 10 μM of LY294002 24 h or transfected with BRCA1-IRIS siRNA first for 48 h then LY294002 treatment; FIG. 3B is expression of the indicated proteins in HOSE/vector or /IRIS treated or not with LY294002 (10 μM) for 24 h; FIG. 3C is expression of indicated proteins in HOSE/IRIS (left) or SK-OV-3 cells after transfection with siLuc or siIRIS (72 h, right); and FIG. 3D is viability of HOSE/vector, /AKT1, /AKT2 cells following transfection with siIRIS or siIRIS+siPTEN. Values represent mean±SD of 24 samples done in 3 different times, where *=p<0.05.

FIGS. 4A-4C show cisplatin triggers BRCA1-IRIS, AKT and survivin expression and activation in HOSE cells wherein FIG. 4A is expression of the indicated proteins in HOSE cells exposed to 0, 5, 10, 30, 50 or 100 μM Cisplatin for 24 h; FIG. 4B is expression of the indicated proteins in HOSE cells or HOSE cells transfected with siIRIS or siAKT for 48 h exposed to 0, 10, 30, 50 μM of cisplatin for 24 h; and FIG. 4C is expression of the indicated proteins in HOSE cells exposed to 0, 5, 10, 30, 50 or 100 μM cisplatin for 24 h.

FIGS. 5A-5D show ectopic or endogenous BRCA1-IRIS overexpression protects ovarian cells from cisplatin-induced survivin down-regulation and cell death wherein FIG. 5A is expression of the indicated proteins in HOSE/vector, /IRIS, /HA-AKT1, 2 or 3 treated with 0, 35 or 70 μM cisplatin for 24 h; FIG. 5B is expression of the indicated proteins in IGROV-1, MCAS, OVCAR-5 or SK-OV-3 cell lines treated with 0, 35 or 70 μM of cisplatin for 24 hrs; FIG. 5C is viability (solid lines) and apoptosis (dashed lines) measurements in cultures identical to (A) treated with increasing concentrations of cisplatin for 48 h; and FIG. 5D is viability (solid lines) and apoptosis (dashed lines) in cultures identical to (B) treated with increasing concentrations of cisplatin for 48 h. In C and D values represent the means of 24 different samples done in 3 separate experiments±SD.

FIGS. 6A-6E show BRCA1-IRIS silencing sensitizes ovarian cells to low cisplatin concentration induced survivin downregulation and cell death wherein FIG. 6A is expression of indicated proteins in HOSE/vector, /AKT1, 2 and /3 cells (A) or MCAS, OVCAR-5 and SK-OV-3 cells; FIG. 6B is transfected or not with BRCA1-IRIS siRNA transfected and treated or not with 0, 35 or 70 μM of CDDP; FIG. 6C is generation of HOSE/Bcl-2 and /Bcl-XL cell lines (left) and expression of indicated proteins in HOSE/Bcl-2 and /Bcl-XL transfected or not with BRCA1-IRIS siRNA and treated 0 or 35 μM of cisplatin (right); FIG. 6D is viability of HOSE/vector, /Bcl-2 or /Bcl-XL transfected with control, AKT, BRCA1-IRIS or survivin siRNAs alone or in combination±35 μM of cisplatin. Value are means of 24 samples in 3 different experiments±SD, *=p<0.05, **=p<0.01 and ***=p<0.001; and FIG. 6E is cell viability measured using MTT assay of MCAS and SK-OV-3 cells transfected or not with BRCA1-IRIS siRNA and treated with 0 or 35 μM of Cisplatin. Values presented are percentage of control of the mean of 24 samples in 3 different experiments±SD, *=p<0.05, **=p<0.01.

FIGS. 7A-7I show p53-dependent and -independent induction of WIP1 by BRCA1-IRIS. FIG. 7A shows expression of BRCA1-IRIS in the breast cancer cell lines BT474 and SKBR3 with p53-mutant or MCF7 with wild-type p53 and HME 2 doxycycline inducible His-BRCA1-IRIS, IRIS2 and IRIS3 and uninducible, IRIS1; FIG. 7B shows expression of BRCA1-IRIS, p53, PPM1D and GAPDH mRNAs in BT474-, MCF7- and SKBR3-silenced cells; FIG. 7C shows HME, IRIS1 or induced IRIS2 and IRIS3; FIG. 7D shows expression of the indicated proteins in BRCA1-IRIS-silenced BT474, MCF7 and SKBR3; FIG. 7E shows HME, IRIS1 and induced IRIS2 and IRIS3; FIG. 7F shows expression of WIP after BRCA1-IRIS silencing in MCF7 cells stably expressing shp53, HEK293T or following infection of SAOS2 cells with His-tagged BRCA1-IRIS cDNA; FIG. 7G shows transactivation assay in HME cells co-transfected with pGL3-hp53, pGL3-hPPM1D1 or pGL3-hCycD1 promoters and vehicle (none), siGFP, siIRIS, pcDNA3 or pcDNA3-IRIS or pcDNA-p53 or pcDNA-WIP1 as indicated; FIG. 7H shows expression of IRIS1, IRIS2 and IRIS3; and FIG. 7I shows lipofectamine expression.

FIGS. 8A-8H show BRCA1-IRIS induces the expression of p53 and WIP1 by a post-transcriptional mechanism. FIG. 8A shows expression of HuR, NF-κB/p65 in total lysates; FIG. 8B shows nuclear (soluble and chromatin-bound) and cytoplasmic extracts from BRCA1-IRIS-silenced MCF7 and SKBR3 cells; FIG. 8C shows HuR expression (green) in MCF7 and SKBR3-silenced cells, blue in DAPI-stained DNA; FIG. 8D shows HuR protein (upper) or PPM1D and p53 mRNAs (lower) in polysomal HuR immunoprecipitates of BRCA1-IRIS-silenced MCF7 and SKBR3 cells; FIG. 8E shows expression of HuR, NF-κB/p65 in total lysates; FIG. 8F shows nuclear (soluble and chromatin-bound) and cytoplasmic extracts from HME or induced IRIS2 and IRIS3; FIG. 8G shows HuR expression (green) in induced IRIS2 and IRIS3, blue is DAPI-stained DNA; and FIG. 8H (left panel) shows immunoprecipitates of polysomal extracts done using HuR or immunoglobulinG (IgG) (control) antibodies; FIG. 8H (right panel) shows HuR protein (upper) or PPM1D and p53 mRNAs (lower) in polysomal HuR immunoprecipitates of HME or induced HME cells; and FIG. 8I shows IRIS expression.

FIGS. 9A-9K show BRCA1-IRIS-induced WIP1 prevents p38MAPK/p53 activation by short-wavelength UV light (UVC) irradiation. FIGS. 9A-9E show expression of indicated proteins in control (FIG. 9A), p53-silenced (FIG. 9B), p38-inactivated (FIG. 9C), BRCA1-IRIS-silenced cells (FIG. 9D) HME or MCF7 cells (FIG. 9E) exposed or not to 20 mJ/cm² UVC; FIG. 9F shows expression of indicated proteins in IRIS1 and induced IRIS3 exposed to increasing doses of UVC; FIG. 9G shows percentage of phosphorylated p53 or p38 compared with total p53 or p38 in IRIS1 or induced IRIS3 exposed to increasing doses of UV; FIG. 9H shows expression of the indicated proteins in HME and induced IRIS3 treated with 20 mJ/cm² of UVC, 10 μM CCT007093 or both (*P<0.05 and ** P<0.001); FIGS. 9I-9K are graphs relating to IRIS expression.

FIGS. 10A-10F show BRCA1-IRIS silencing enhances the killing of HME cells by geno-/cell-toxic stresses, wherein FIGS. 10A-10D show percentage of dead cells in control, BRCA1-IRIS-silenced, BRCA1-IRIS-silenced/p38-inactivated or BRCA1-IRIS silenced/p53 silenced MCF7 cells treated with (FIG. 10A) nothing, (FIG. 10B) 40 mJ/cm², (FIG. 10C) 10 μM etoposide or (FIG. 10D) 15 μM H₂0₂ as detected by LIVE/DEAD analysis; FIG. 10E shows levels of cleaved poly-(ADP ribose) polymerase in control and BRCA1-IRIS-silenced MCF7 and SKBR3 cells after none, 10 μM etoposide or 15 μM H₂0₂ treatment (*P<0.05 and **P<0.001); and FIG. 10F comprises panels A-X, showing gene expression, in accordance with the present invention.

FIG. 11A shows BRCA1-IRIS overexpression protects in a WIP1-dependent manner HME cells against geno-/cell-toxic stresses-induced cell death. Number of TUNEL+cells in HME or induced IRIS3 after exposure to 40 mJ/cm² UVC, 10 μM etoposide or 15 μM H₂0₂ (*P<0.05 and **P<0.001); and FIG. 11B is a graph showing IRIS expression, in accordance with the present invention.

FIGS. 12A and 12B show BRCA1-IRIS transforms HME cells in combination with WIP1 or oncogenic Ras (Ras^{V12}). FIG. 12A shows the number of cells in IRIS1 and induced IRIS2 or IRIS3 transfected with nothing, pcDNA3-Ras^{V12} or pcDNA3-WIP1. Data presented as mean±s.d. from three separate experiments done in triplicates. FIG. 12B shows the number of colonies of IRIS1 and induced IRIS3 transfected with pcDNA3-Ras^{V12} or pcDNA3-WIP1 growing in soft agar coated wells. Data presented as mean±s.d. from three separate experiments done in triplicates. +P≦0.5 compared with IRIS1, wherein, in FIGS. 12A and 12B, *P<0.05 and **P<0.001. FIG. 12C shows expression of IRIS, in accordance with the present invention.

FIG. 13 is a table with data for cell death in BRCA1-IRIS silenced cells, in accordance with the present method.

### DETAILED DESCRIPTION OF THE INVENTION

Determination of patients at risk of developing aggressive cancer is based on a discovery that the amount of BRCA1-IRIS is directly linked to anti-apoptosis in cancer cells. Prior to the present discovery, one difficulty in assessing whether a primary tumor will metastasize was indeterminate, due to a lack of any metastatic markers that could be detected in primary tumor cells as an indication of possible future metastatic growth of that primary tumor. However, the present discovery that BRCA1-IRIS is a marker, i.e. oncogene, that induces metastasis when overexpressed allows one to now identify patients as possible risk candidates for developing aggressive cancer. For example, overexpression of BRCA1-IRIS in tumors is possible, as early as ductal carcinoma MC2 (DCIS) or earlier, in the genesis of breast cancer or other cancers. As a result, one can analyze a biopsy from tumors, such as breast cancer cells or other suitable tumor cells, in patients to identify patients who are at a greater risk of developing metastatic cancer, including metastatic breast cancer or other cancers.

One advantageous method for detecting BRCA1-IRIS overexpression includes the development of a BRCA1-IRIS monoclonal antibody which specifically recognizes an epitope of the BRCA1-IRIS protein, namely an intron 11 unique portion of BRCA1-IRIS. This antibody will be deposited in accordance with the Budapest Treaty, having accession no. ______. All restrictions to the deposited sample will be lifted upon granting of any patent in this application and the biological sample deposited will be replenished if the deposited sample is no longer viable.

A therapeutic method of reducing the growth or proliferation of cancer cells is provided by administering a therapeutically effective amount of an inhibitor of BRCA1-IRIS to cancer cells to thereby reduce the growth or proliferation of cancer cells. In one advantageous form, small interference RNA (siRNA) sequence induces BRCA1-IRIS mRNA degradation in cells expressing either normal or high levels of BRCA1-IRIS mRNA. This leads to a decrease in the overall BRCA1-IRIS protein level and the oncogenic properties which overexpression of BRCA1-IRIS induces in tumor cells.

The siRNA converts tumor cell into less transformed cells or kills the cells. In one advantageous form, the siRNA sequence can be placed in a viral siRNA vector to produce a gene therapy approach. The vector is introduced to cells, such as cancer or tumor cells, resulting in a transformation of the cancer cells to have reduced levels of BRCA1-IRIS expression, thereby reducing a burden of cancer and possibility of metastisizing cancer.

The preferred dose for administration of an the inhibitor of BRCA1-IRIS, in accordance with the present invention is that amount which will be effective in limiting the growth or proliferation of cancer, or increase the apoptosis of cancer cells, of by lowering or inhibiting BRCA1-IRIS expression, and one would readily recognize that this amount will vary greatly depending on the nature and extent of the disease and the condition of a patient. An “effective amount” of the inhibitor to be used in accordance with the invention is intended to mean a nontoxic but sufficient amount of the agent, such that the desired prophylactic or therapeutic effect is produced. Thus, the exact amount of the inhibitor that is required will vary from subject to subject, depending on the species, age, and general condition of the subject, the severity of the condition being treated, the particular carrier or adjuvant being used and its mode of administration, and the like. Similarly, the dosing regimen should also be adjusted to suit the individual to whom the composition is administered and will once again vary with age, weight, metabolism, etc. of the individual. Accordingly, the “effective amount” of any particular inhibitor, e.g. an siRNA to BRCA1-IRIS mRNA or an antibody to BRCA1-IRIS protein, will vary based on the particular circumstances, and an appropriate effective amount may be determined in each case of application by one of ordinary skill in the art using only routine experimentation.

Using this monoclonal antibody, along with an immunohistochemical protocol, to stain BRCA1-IRIS present in a native biological sample, as well as a paraffin embedded tumor sample, allows one to quantify the level of BRCA1-IRIS in a biopsy and to determine the expression level of BRCA1-IRIS. Based on statistics of BRCA1-IRIS expression, one can determine whether a patient is at a risk of developing aggressive cancer if the expression exceeds a threshold amount. For example, one can correlate the expression level of BRCA1-IRIS in patients with aggressive cancer to thereby determine what a threshold amount of overexpression of BRCA1-IRIS is which indicates a patient is a risk candidate for developing aggressive cancer.

In one example of quantifying the amount of IRIS, human tumors were fixed in formalin within 8-48 h from resection then embedded in paraffin. Air-dried for 30 min (minimum), heated to 60° C. in oven for 20 min, cooled to room temperature in ˜0 min, before de-paraffinizing by incubating 2 times in Xylenes 5 min each, rehydrated by incubating 2 times in 100%, 2 times in 95%, 1 time in 80% and 1 time in 50% ethanol for 4 min each, washed in water for 4 min Epitope retrieval for BRCA1-IRIS was the done by incubating in 10 μM pepsin in 37° C. for 30 min. On an automated system (DAKO Autostainer) slides were exposed to 3% hydrogen peroxide for 5 min, followed by incubation with 1° (mouse anti-human BRCA1-IRIS) antibody for 30 min, followed by several washes with Tris-buffered saline (TBS) then incubation with 2° (goat anti-mouse IgG) antibody for 30 min Slides were then developed with 3,3′-diaminobenzidine (DAB) for 5 min, counterstained with Meyer's hematoxylin for 5 min and cover-slipped.

Scoring for immunohistochemical staining of the slides. For human samples, positive controls of several un-diseased tissues (kidney, liver, placenta, spleen and normal breast) and negative controls with 1° antibody replaced with TBS or antibody depletion by incubating with antigen before staining were all done. Nuclear staining in more than 10% of tumor cells was considered positive and staining intensity was determined based on ASCO/CAP guidelines. Strong nuclear (chromatin at nuclear periphery) for BRCA1-IRIS in >30% of invasive carcinoma cells=3+; in ≧10% of invasive tumor cells=2+; weak and incomplete nuclear BRCA1-IRIS staining in invasive tumor cells=1+, no staining=0. Tumors with 0 and 1+ staining were considered negative and cases scored as 2+ equivocal, and 3+ were considered positive, evaluated on 4× and 10× magnifications, blindly by two pathologists.

Statistical analysis. Comparisons of statistical differences were done using the Student t-test for paired and unpaired data. Statistical significance was assumed at a P-value of ≦0.05. To compare multiple groups with one control group, analysis of variance (ANOVA) was used.

In addition, one can use PCR to amplify the amount of BRCA1-IRIS mRNA present in a sample to thereby determine the amount of BRCA1-IRIS expression in a patient. The following is one method in which one can use PCR to quantify the amount of BRCA1-IRIS.

Total RNAs from tumor cells were isolated with Trizol (Gibco, Life Technologies) and DNaseI-treated. Routinely we use SuperScript One-Step RT-PCR with Platinum Taq (Invitrogen) to amplify the RNA (using 500 ng or less) as template in the one step in the RT/PCR experiments RNA. Using the primer set “Forward primer: 5′-TTCTTCCAAA CAAATGAGGC ATCAGTCTGA-3′ (SEQ ID NO:1), Reverse primer: 5′-GTTCCTTTAA CTATACTTGG AAATTTGTAA AATGTGCTCC-3′ (SEQ ID NO:2) that amplifies ˜200 bp of BRCA1-IRIS RNA 3′ end. Comparisons of statistical differences were done using the Student t-test for paired and unpaired data. Statistical significance was assumed at a P-value of ≦0.05. To compare multiple groups with one control group, analysis of variance (ANOVA) was used.

The present method will now be described with reference to specific examples, as follows, using the described protocols.

Materials and Methods

Cell culture. OV-90, OVCAR-3 and SK-OV-3 were obtained from ATCC, IGROV-1, OVCAR-5, OVCAR-8, OVCAR-420 and MCAS were a gift from Drs. S. Mok or R. Drapkin (Harvard Medical School) Immortalized ovarian surface epithelial cell line (HOSE1) was kindly provided by Dr. Nelly Auersperg (University of British Columbia, Canada), and HOSE2 was a gift from Dr. S. Mok (Harvard Medical School). These are normal HOSE cells immortalized with SV40 large T antigen, were grown in medium containing: 45% Medium 199, 45% MCDB105 and 10% fetal bovine serum (FBS, HyClone). All other cell lines were grown in RPMI 1640 (Invitrogen) supplemented with 10% FBS. Where indicated cisplatin/CDDP dissolved in DMSO (Novaplus, Ben Venus Laboratories, Inc.) was added.

HOSE cell lines generation. Drs. Sellers and Korsmyer (Dana-Farber Cancer Institute), Abbas (University of California, San Francisco), and Alteri (University of Massachusetts) supplied us with the retroviral expressing vectors HA-tagged Myr-AKT1, 2 and 3, Bcl-2, -XL and survivin, respectively. Lentivirus BRCA1-IRIS expressing vector was constructed by RT/PCR cloning into plenti/V5-D-TOPO vector (Invitrogen). Recombinant viruses produced in 293T cells were immediately added to cells and cell lines were selected with appropriate antibiotics.

siRNA transfection. BRCA1-IRIS siRNA was described previously (30). AKT siRNA (#6211) that targets AKT1 and 2 (not 3), and survivin siRNA (#6351) were purchased from Cell Signaling Technology. Cells were transfected with Oligofectamine (Invitrogen) according to manufacturer's instructions.

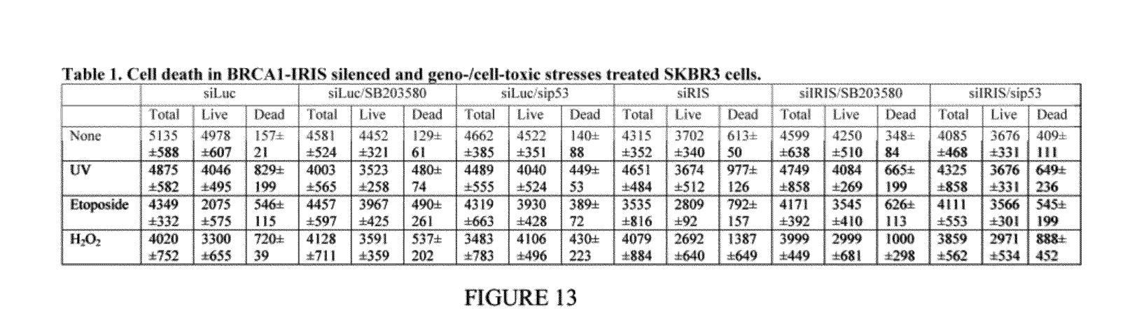

Quantitative Real-time RT/PCR. Real-time RT/PCR for BRCA1-IRIS, AKT1, AKT2, AKT3, BRCA1/p220 and survivin and several housekeeping genes (see the table in FIG. 13, which was done using iScript One-Step RT-PCR kit with SYBER® Green (Bio-Rad), according to manufacturer's instructions).

Immunoblotting. Cells treated with different concentrations of cisplatin or not were collected in PBS, sonicated 3 times, cleared by centrifugation at 10,000 g, 10 min at 4° C., IB'd using antibodies for survivin (#2802), AKT isoforms sampler kit (#9940, which contains total and p-S473/T308-specific antibodies), p-GSK3β (S9)-specific antibody (#9336), PTEN (D4.3) XP™ (#9188), FOXO1 (L27, #9454) and FOXO3a (#9467) were from Cell signaling. Bcl-2 (clone 124; Dako Diagnostics AG), Bcl-xL (Transduction Laboratories), actin (Ab-1, CP01, Oncogene) and phospho-Survivin (T34, Novus Biologicals). Mouse antibody against BRCA1-IRIS was generated in our laboratory.

MTT and activated caspases 3 and 7 assays. Done using the Cell Titer 96® AQᵤₑₒᵤₛ One Solution Cell Proliferation Assay (#G3580, Promega) Caspase-Glo 3/7 Assay (#G8091, Promega), respectively as per manufacturer's instructions. Measurements were obtained using optical at 490 nm. Each experiment was done in 8 samples and the whole experiment was repeated 3 separate times.

Cell cycle analysis. Cell cycle analysis was carried out by flow cytometery after PI staining using standard protocol.

Results

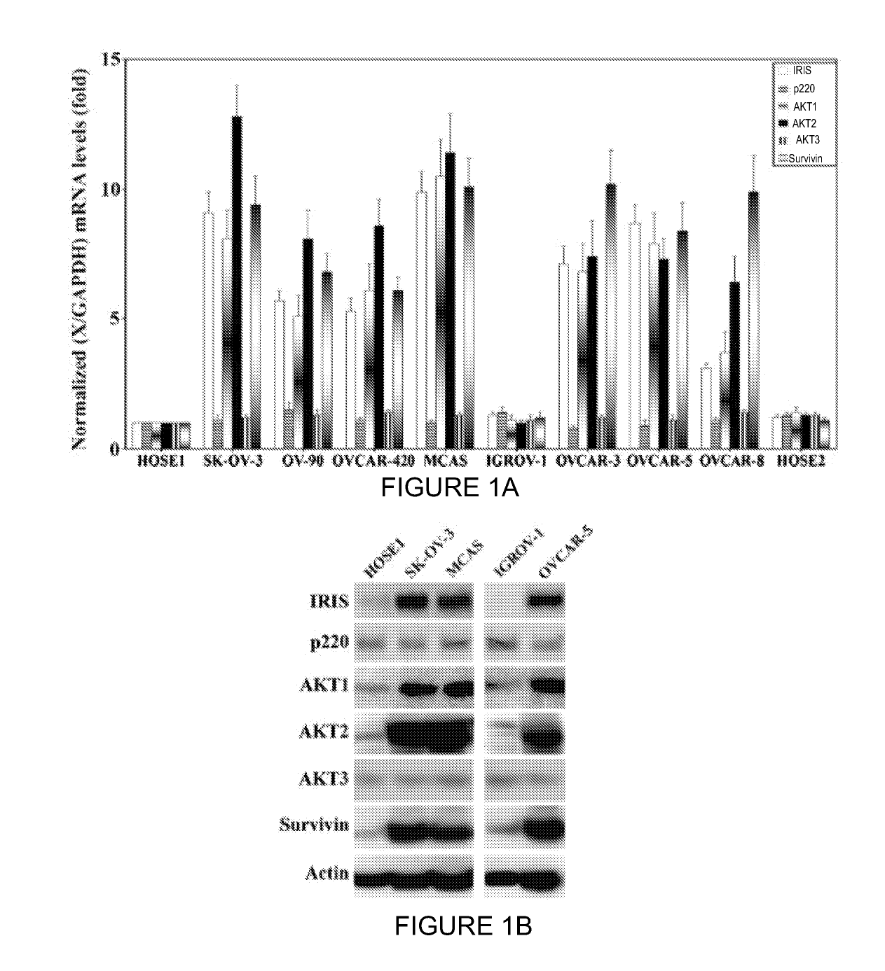

BRCA1-IRIS, AKT1, 2 and survivin are overexpressed in ovarian cancer cell lines. Using total RNAs and proteins from 2 immortalized human surface epithelial cell lines (HOSE1 and 2) and several ovarian cancers cell lines, we show using real-time RT/PCR and immunoblotting (IB) that all ovarian cancer cell lines, except IGROV-1 express higher levels of BRCA1-IRIS mRNA and protein, respectively, while normal levels of BRCA1/p220 mRNA and protein (FIGS. 1A and B). Since AKT and survivin are important inducers of drug-resistance in ovarian cancer cells, we examined the levels of AKT (isoforms 1, 2 and 3) and survivin mRNAs and proteins in these ovarian cancer cells lines. AKT1, AKT2 (not AKT3) and survivin mRNAs and proteins were also high in all ovarian cancer cell lines, except IGROV-1 (FIGS. 1A and B), implying co-overexpression of BRCA1-IRIS, AKT and survivin in some ovarian cancer cell lines.

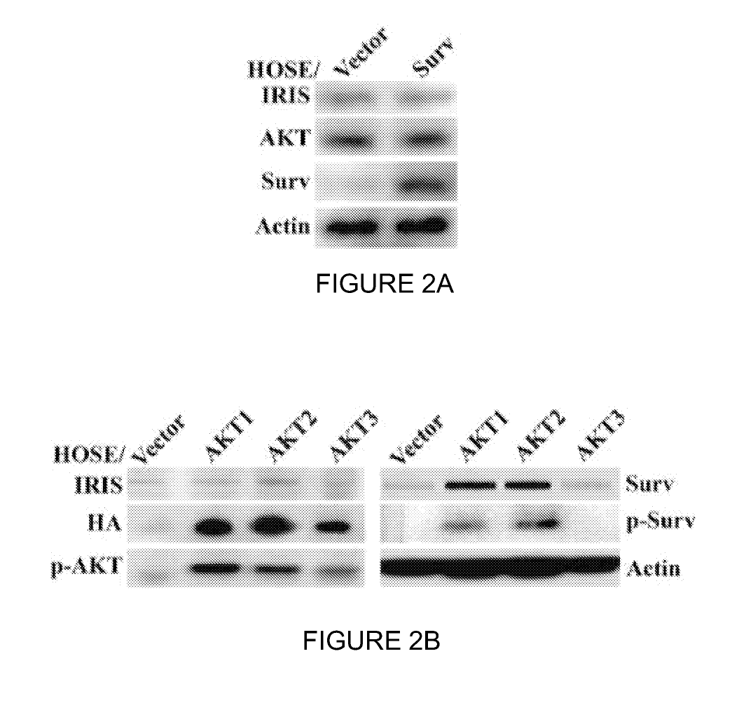

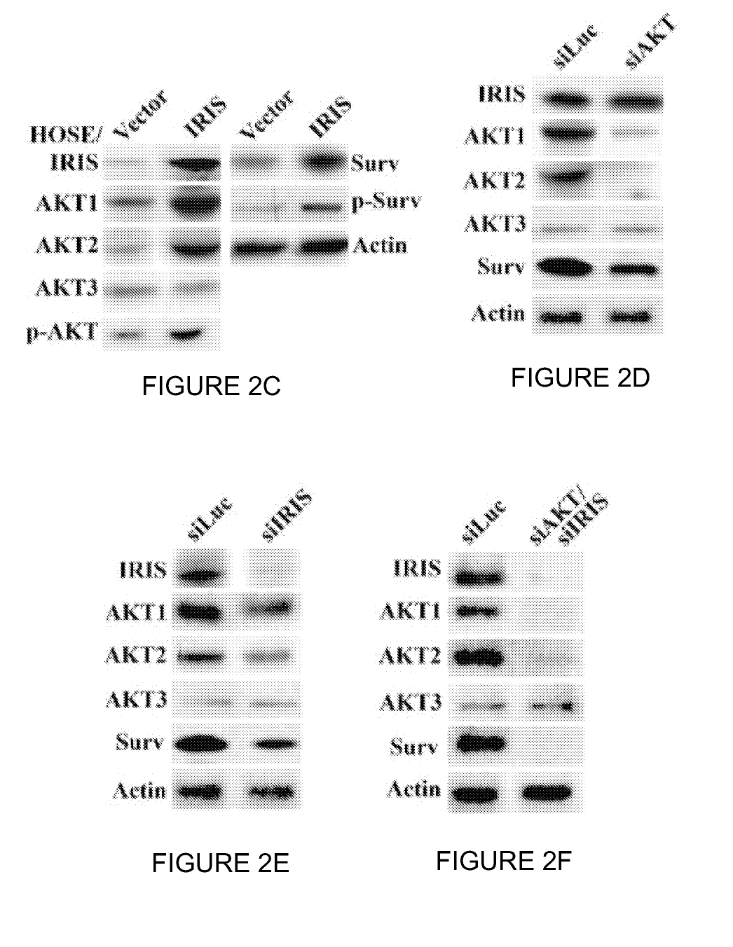

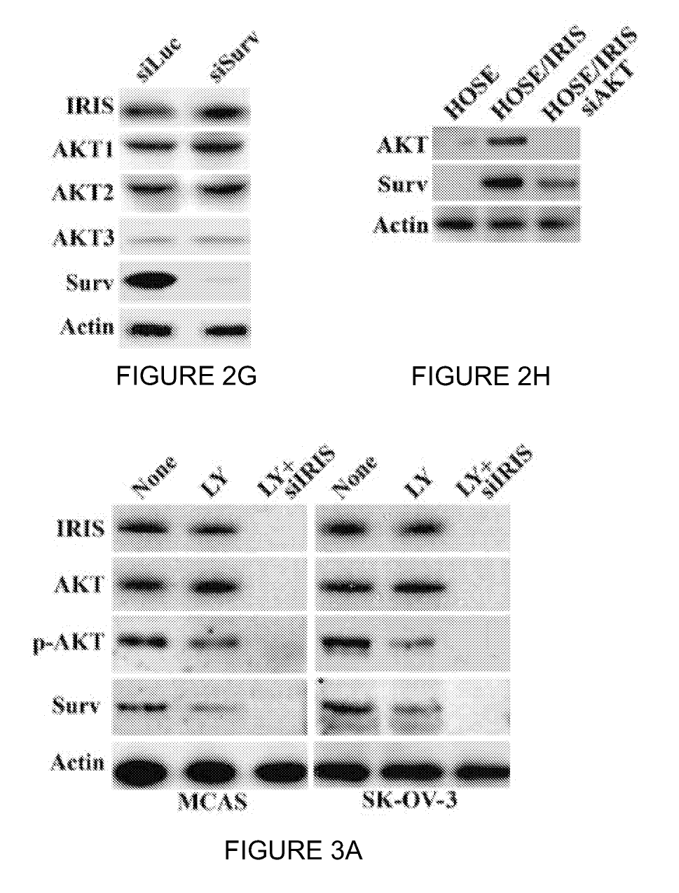

Ectopic BRCA1-IRIS expression triggers AKT and survivin expression and activation. To test the hypothesis that BRCA1-IRIS triggers AKT/survivin expression in ovarian cells, HOSE1 and 2 cell lines stably expressing BRCA1-IRIS (HOSE/IRIS), myristoylated-HA-tagged-AKT1, -AKT2, -AKT3 (HOSE/AKT1, /2 and /3) or survivin (HOSE/survivin) were generated. The data in FIGS. 2A-C represent results obtained using cell lines generated in HOSE1 cells, however, identical results were obtained using cell lines generated in HOSE2.

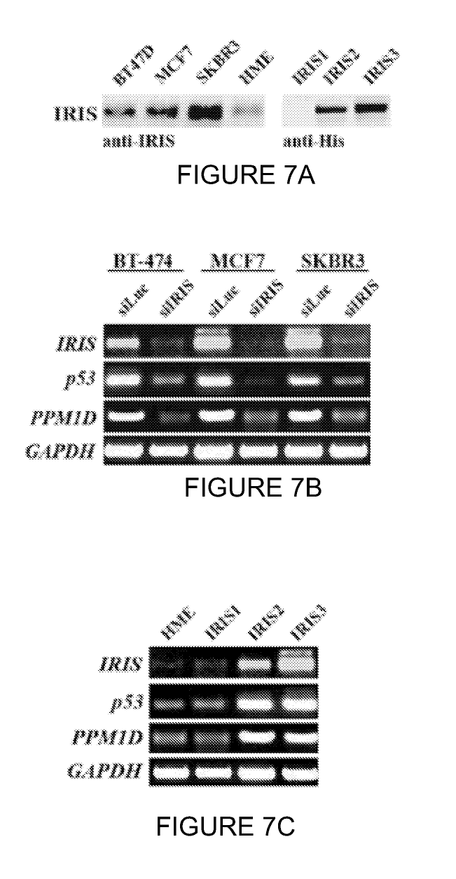

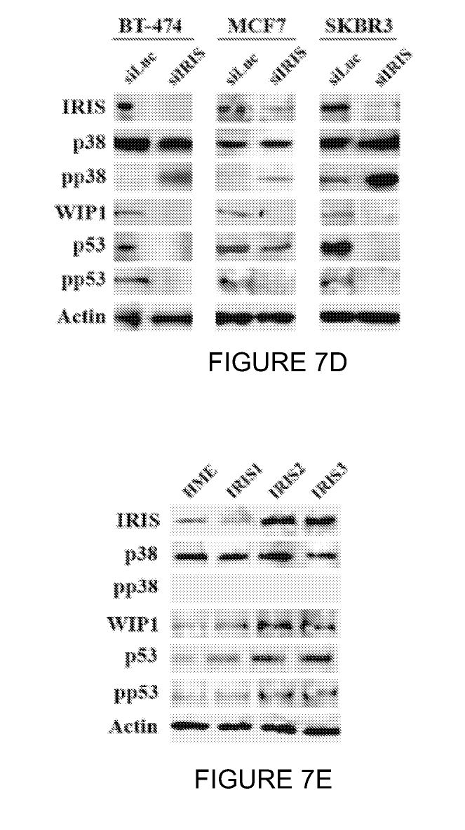

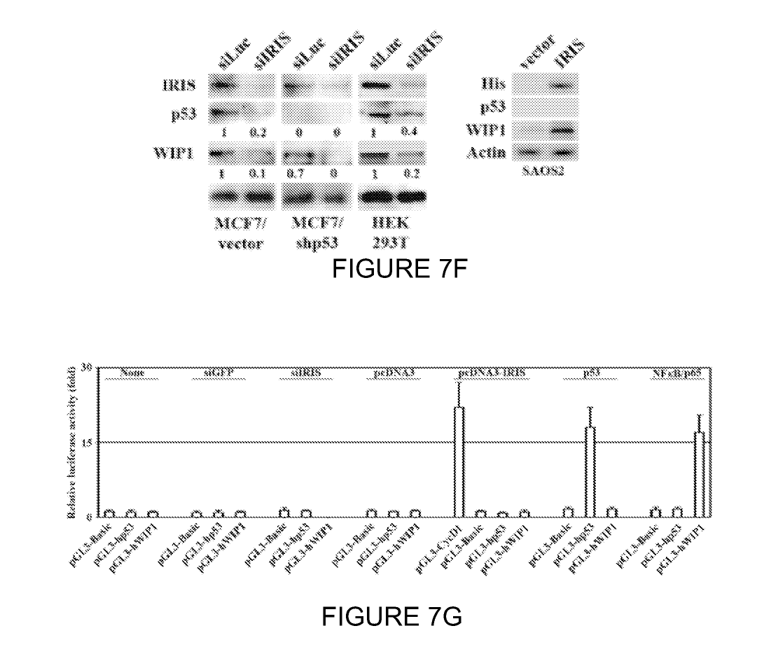

Compared to control, HOSE/survivin showed normal levels of BRCA1-IRIS and total AKT (detected using pan-antibody, FIG. 2A). HOSE/AKT1, /AKT2 and /AKT3 showed normal level of BRCA1-IRIS (FIG. 2B) but high levels of p-AKT [detected using a mixture of anti-phosph-T308 and -S473 antibodies], survivin and p-survivin [detected using anti-phosph-T34 antibody] (FIG. 2B). HOSE/IRIS, on the other hand, showed high levels of AKT1 and AKT2 (not AKT3), p-AKT, survivin and p-survivin (FIG. 2C). Increase in AKT1, AKT2 (not AKT3) and survivin mRNAs levels was also detected following BRCA1-IRIS overexpression in HOSE1 and 2 cell lines (FIG. 7H). Taken together, this data provides evidence that BRCA1-IRIS overexpression triggers AKT1, AKT2 and survivin expression and activation.

BRCA1-IRIS-induced survivin expression is partially AKT-dependent. AKT (siRNA silences AKT1 and AKT2), BRCA1-IRIS, survivin or AKT and BRCA1-IRIS were silenced in SK-OV-3 cells (endogenously overexpressing all proteins, see FIG. 1B) for 72 h. Compared to control, AKT silencing had no effect on BRCA1-IRIS expression, but partially reduced survivin expression (FIG. 2D, perhaps due to BRCA1-IRIS related effect). BRCA1-IRIS silencing reduced the expression of AKT1, AKT2, and also only partially reduced survivin expression (FIG. 2E, perhaps due to an AKT related effect). Simultaneous silencing of BRCA1-IRIS and AKT, like survivin silencing completely abolished survivin expression (FIGS. 2F and 2G). Survivin silencing had no effect on BRCA1-IRIS or AKTs the expression (FIG. 2G). Identical results were obtained using MCAS cell line. Moreover, the levels of AKT1, 2 (not 3) and survivin mRNAs also decreased in SK-OV-3 and MCAS cells depleted from BRCA1-IRIS (FIG. 7I). Furthermore, AKT silencing in HOSE/IRIS cells (siRNA targets AKT1 and 2) for 72 h, while completely abolished AKT expression, it only partially decreased survivin expression (FIG. 2H). These data provide evidence that BRCA1-IRIS-induced survivin expression is partially AKT-dependent.

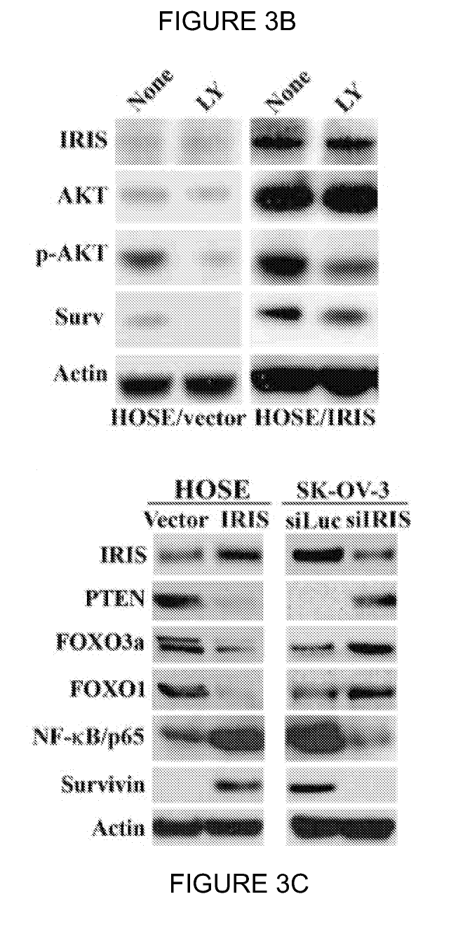

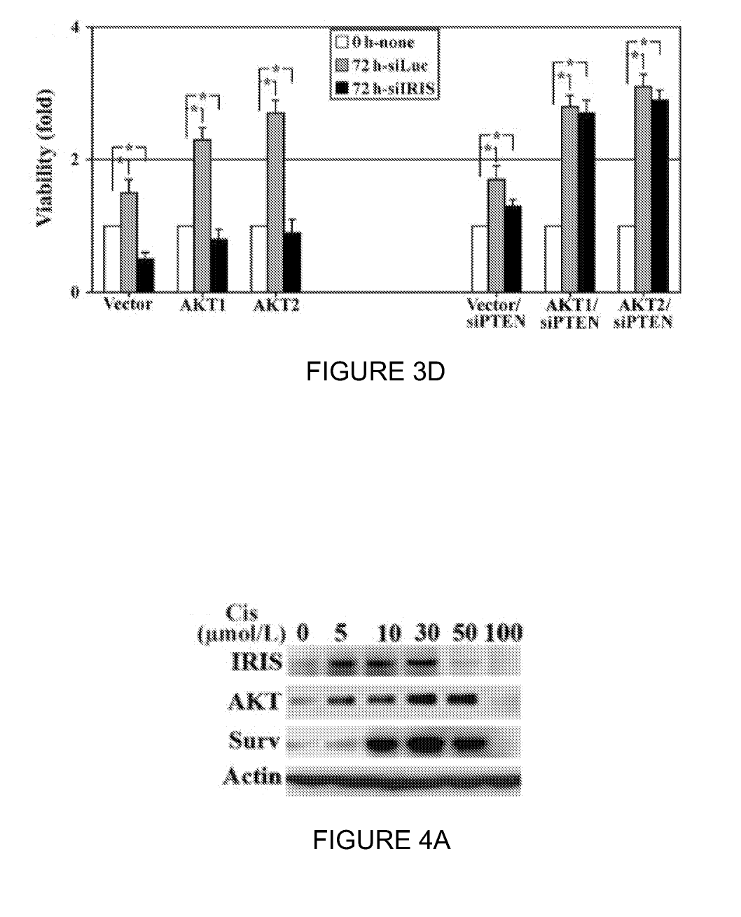

BRCA1-IRIS overexpression-induced survivin expression is partially PI3′K/AKT-independent. To study this further, MCAS or SK-OV-3 cells were exposed to the PI3′K inhibitor, LY294002 for 24 h or were transfected with BRCA1-IRIS siRNA for 48 h before they were exposed to LY294002 and left for another 24 h. Compared to untreated cells, LY294002 alone while did not change the level of total AKT, it decreased the level of p-AKT (FIG. 3A) and survivin in both cell lines (FIG. 3A). In contrast treatment of BRCA1-IRIS silenced cells with LY294002 completely abolished survivin expression (FIG. 3A). These data provides evidence that BRCA1-IRIS-induced survivin expression is partially PI′3K/AKT-independent. Indeed, LY294002 decreased survivin expression in HOSE/vector cells but had little effect in HOSE/IRIS cells (FIG. 3B). Furthermore, data collected show that silencing of BRCA1-IRIS in HOSE/AKT1 or 2 cells (expression driven from heterologous promoter and proteins are myristoylated/activated) reduces the expression of the exogenous proteins as measured by HA immunoblotting.

Finally, in chromatin immunoprecipitation (ChIP) experiments using HOSE1 or SK-OV-3 although BRCA1-IRIS was bound to the Cyclin D1 promoter (known BRCA1-IRIS target, 35), it was not bound to AKT1, AKT2 or survivin promoter (data not shown). Moreover, in co-transfection experiments, BRCA1-IRIS overexpression did not activate transcription from AKT1, AKT2 and survivin promoter driven reporter plasmids (data not shown). Taken together, the data provides evidence that BRCA1-IRIS-induced AKT1 and AKT2 is not transcriptional but post-transcriptional driven (e.g., promotes their mRNA and/or protein stabilities, see 38), and that survivin expression is partially induced by BRCA1-IRIS-dependent/PI3′K/AKT-independent mechanism.

BRCA1-IRIS overexpression induces survivin expression through suppressing PTEN/FOXO1 and FOXO3a expression. Besides inducing the expression of the known surviving transcription inducer, NF-κB (FIG. 3C, see also 37, 38), BRCA1-IRIS overexpression in HOSE cells also reduced the expression of PTEN and its down-stream survivin transcription suppressors, FOXO1 and FOXO3a (39, FIG. 3C). Moreover, BRCA1-IRIS silencing in SK-OV-3 (FIG. 3C) or MCAS (not shown) cells derepressed the expression of these three proteins (FIG. 3C). It is possible that BRCA1-IRIS induces survivin expression by inducing AKT expression and activation or by suppressing PTEN/FOXO1 and/or FOXO3a expression. Indeed, when BRCA1-IRIS was silenced in equal number of HOSE/vector, /AKT1 or /AKT2 cells, the increase in cell number observed following AKT1 and AKT2 overexpression was dramatically reduced (gray bars, FIG. 3D). Co-silencing of PTEN restored the increase in cell number in AKT1 and AKT2 overexpressing cells (black bars, FIG. 3D), suggesting that BRCA1-IRIS silencing kill cells even when AKT1 or 2 are overexpressed, most likely due to increase in PTEN/FOXO1 and FOXO3a expression leading to survivin expression decrease.

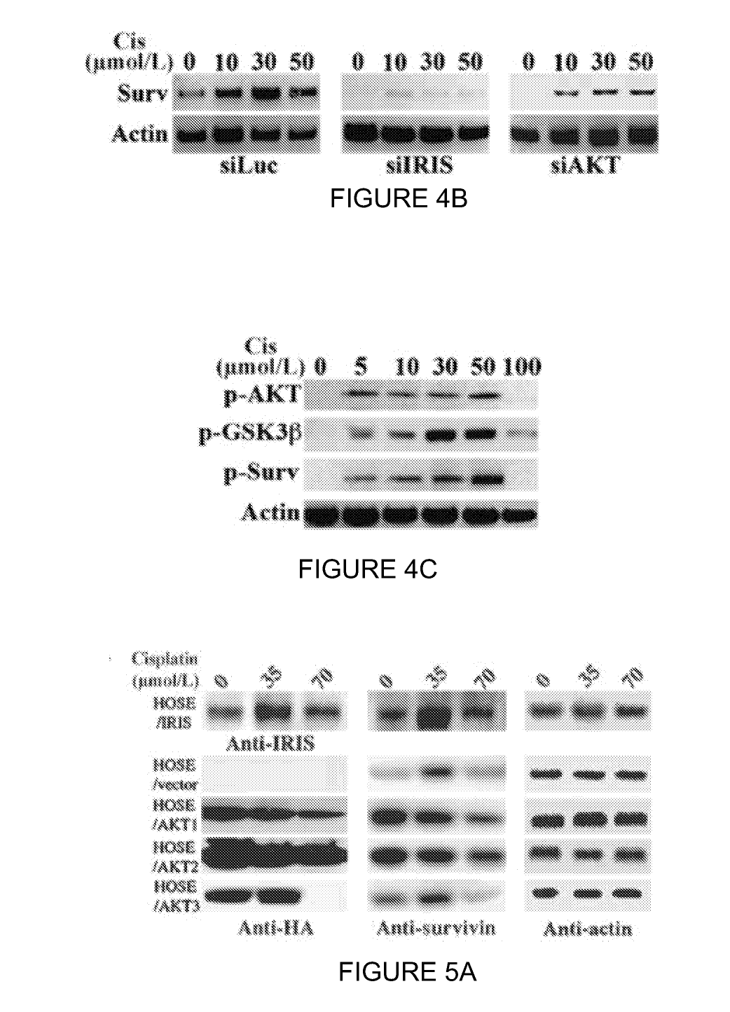

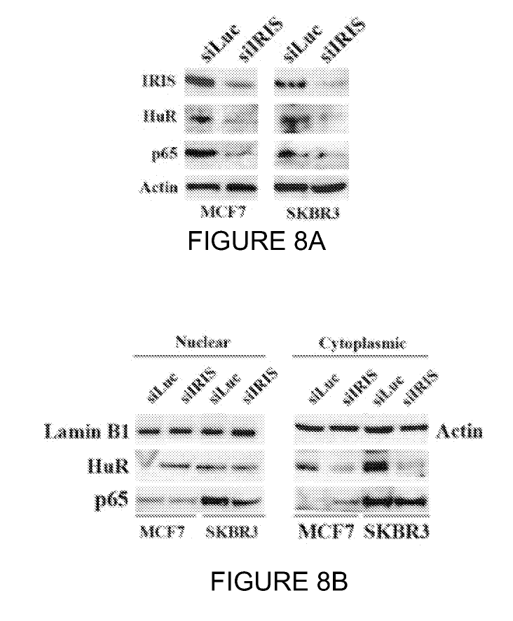

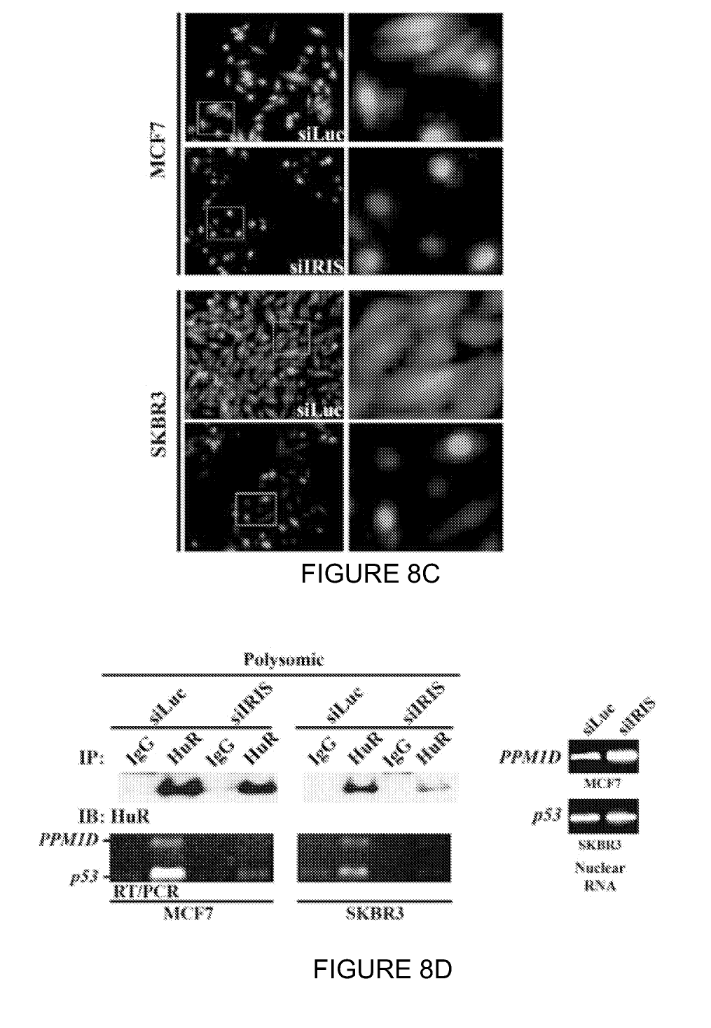

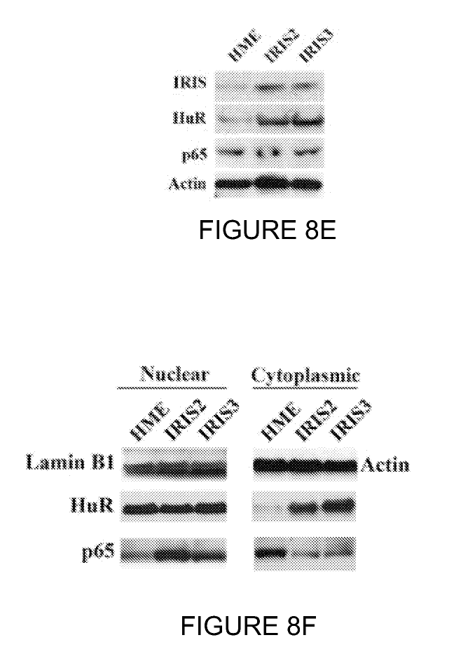

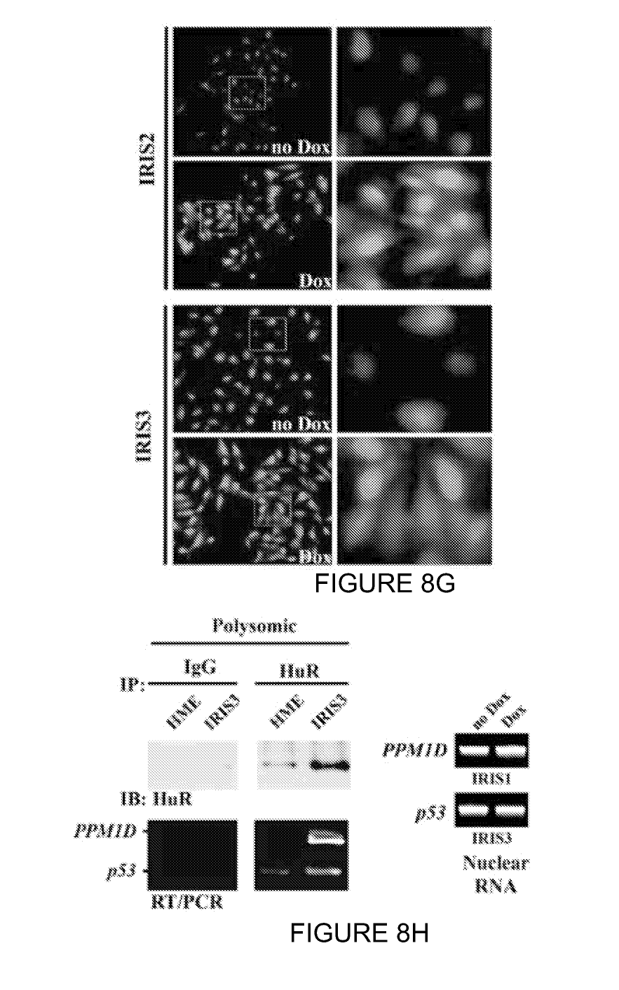

Low cisplatin concentrations trigger BRCA1-IRIS-AKT-survivin expression in HOSE cells. Ovarian cancer cells expressing high level of survivin usually are cisplatin-resistant. To investigate whether BRCA1-IRIS overexpression induces acquired cisplatin resistance, HOSE cells were treated with increasing concentrations of cisplatin and BRCA1-IRIS, AKTs and survivin expression was investigated. Compared to untreated cells, low concentrations cisplatin (≦50 μM) triggered BRCA1-IRIS, AKT and survivin expression (FIG. 4A), while high concentrations (>50 μM) abolished their expression (FIG. 4A). In FACScan analysis no G2/M arrest was detected in HOSE cells treated with any of the cisplatin concentrations used (survivin expression increases in G2/M cells, FIG. 8I). Thus low cisplatin concentrations-induced survivin expression must be due to the induction in AKT and/or BRCA1-IRIS expression by the same treatment.

Interestingly, while silencing of BRCA1-IRIS in HOSE cells completely abolished survivin induction by low cisplatin concentrations (FIG. 4B), AKT silencing only partially blocked survivin induction by low cisplatin concentrations in HOSE cells (FIG. 4B), re-enforces the view that BRCA1-IRIS-induced survivin expression is partially PI3′K/AKT-independent (see above). Finally, AKT and survivin were also activated by low cisplatin concentrations treatment, since we observed phosphorylation of AKT on T308/S437, of its downstream target, GSK-3β on S9 (see 40, FIG. 4C), and of survivin on T34 (FIG. 4C, an event that occurs by cdc2 in viable and proliferating cells, see 41).

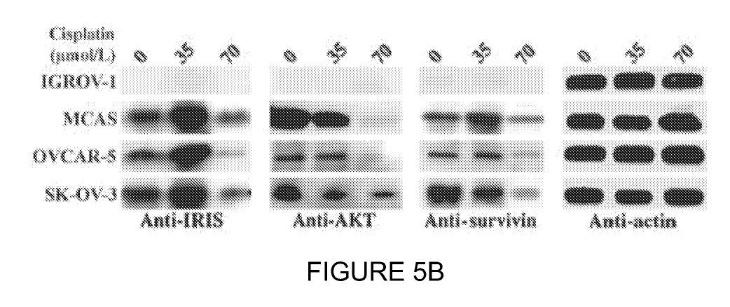

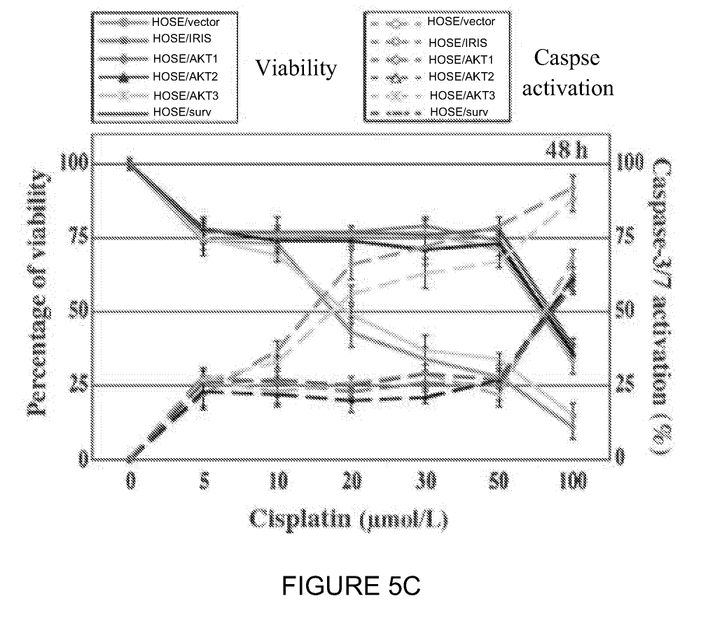

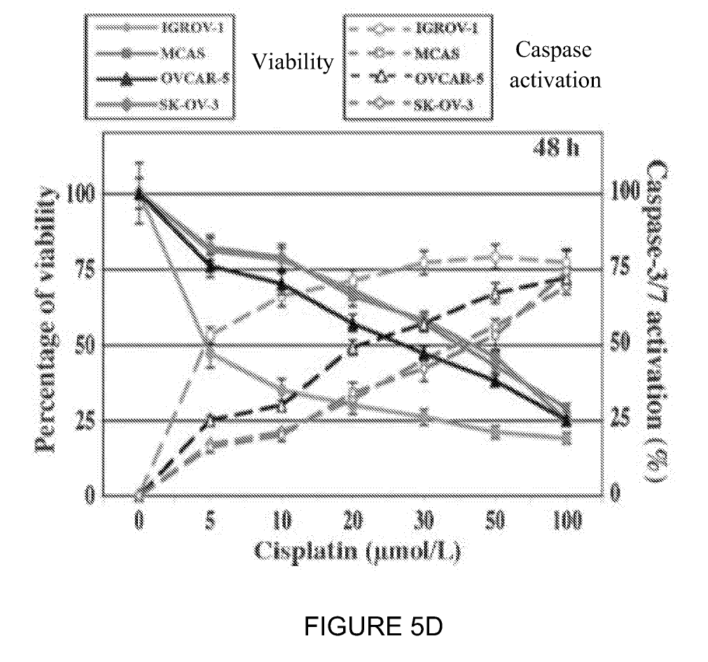

Ectopic BRCA1-IRIS expression protects HOSE cells from cisplatin-induced cell death. HOSE/vector, /IRIS, /AKT1, /AKT2 or /AKT3 were untreated or treated with low (35 μM) or high (70 μM) concentration of cisplatin for 24 h and the expression of survivin was measured by immunoblotting. In untreated HOSE/IRIS, /AKT1 and /AKT2 survivin levels were higher compared to HOSE/vector cell (FIG. 5A). As expected treatment of HOSE/vector and HOSE/AKT3 cells with 35 μM cisplatin increased survivin expression slightly, while 70 μM cisplatin did not (FIG. 5A). In HOSE/IRIS cells, 35 μM and not 70 μM cisplatin increased BRCA1-IRIS and survivin expression (FIG. 5A). In HOSE/AKT1 and HOSE/AKT2 cells, 35 μM cisplatin did not increase survivin expression and 70 μM actually decreased it slightly (FIG. 5A). It is possible that BRCA1-IRIS and to a lesser extent AKT1 and AKT2 overexpression protects survivin from cisplatin-induced downregulation.

Furthermore, HOSE/vector, /IRIS, /AKT1, /AKT2, /AKT3 and /survivin were plated at equal numbers and were incubated with 0, 5, 10, 20, 30, 50 or 100 μM cisplatin for 48 h followed by measurement of cell viability using MTT assay (solid lines, FIG. 5C) or cell death using activated caspase3/7 assay (dashed lines, FIG. 5C). HOSE/vector or HOSE/AKT3 cells were sensitive to high cisplatin concentrations, with IC₅₀ of 15-20 μM (FIG. 5C) that coincided with an increase in activated caspase3/7 (FIG. 5C). HOSE/IRIS, /AKT1, /AKT2 or /survivin were resistant to high cisplatin concentrations-induced cell death and the IC₅₀ was ˜75 μM in these cell lines (FIG. 5C) with low yet measurable levels of active caspase3/7 was detected (FIG. 5C). Taken together, BRCA1-IRIS overexpression protects HOSE cells from high cisplatin concentrations-induced cell death by protecting survivin from cisplatin-induced downregulation.

Endogenous BRCA1-IRIS overexpression protects ovarian cancer cells from cisplatin-induced cell death. IGROV-1, MCAS, OVCAR-5 and SK-OV-3 cell lines were treated with 0.35 μM or 70 μM of cisplatin for 24 h. IGROV-1 cells showed no detectable expression of BRCA1-IRIS, AKT or survivin proteins even after treatment with 35 or 70 μM of cisplatin (FIG. 5B). In contrast, untreated MCAS, OVCAR-5 and SK-OV-3 showed high levels of BRCA1-IRIS, AKT and survivin proteins (FIG. 5B). Treatment with 35 μM of cisplatin increased BRCA1-IRIS in all cell lines (FIG. 5B), did not affect AKT expression in all cell lines (FIG. 5B) and increased (MCAS), had no effect on (OVCAR-5) or decreased (SK-OV-3) survivin expression (FIG. 5B). BRCA1-IRIS, AKT and survivin levels were significantly reduced by 70 μM of cisplatin in all cell lines (FIG. 5B).

Identical numbers of these 4 cell lines were incubated with 0, 5, 10, 20, 30, 50 or 100 μM of cisplatin for 48 h followed by MTT and active caspase3/7 assays. As expected, low viability and high level of active caspase3/7 were detected in IGROV-1 (FIG. 5C), showing an IC₅₀ of ˜5 μM. On the other hand, MCAS, OVCAR-5 and SK-OV-3 cells expressing high level of BRCA1-IRIS-AKT-survivin were more resistant to cisplatin-induced activated caspase3/7 and cell death (FIG. 5C) and showed IC₅₀ of ˜25 μM for OVCAR-5 and ˜40 μM for MCAS and SK-OV-3 cell lines. Taken together these data provide evidence that higher level of BRCA1-IRIS-AKT1/2 induces persistence survivin expression in the face of high concentrations of cisplatin and that protects ovarian cancer cells against cisplatin-induced cell death.

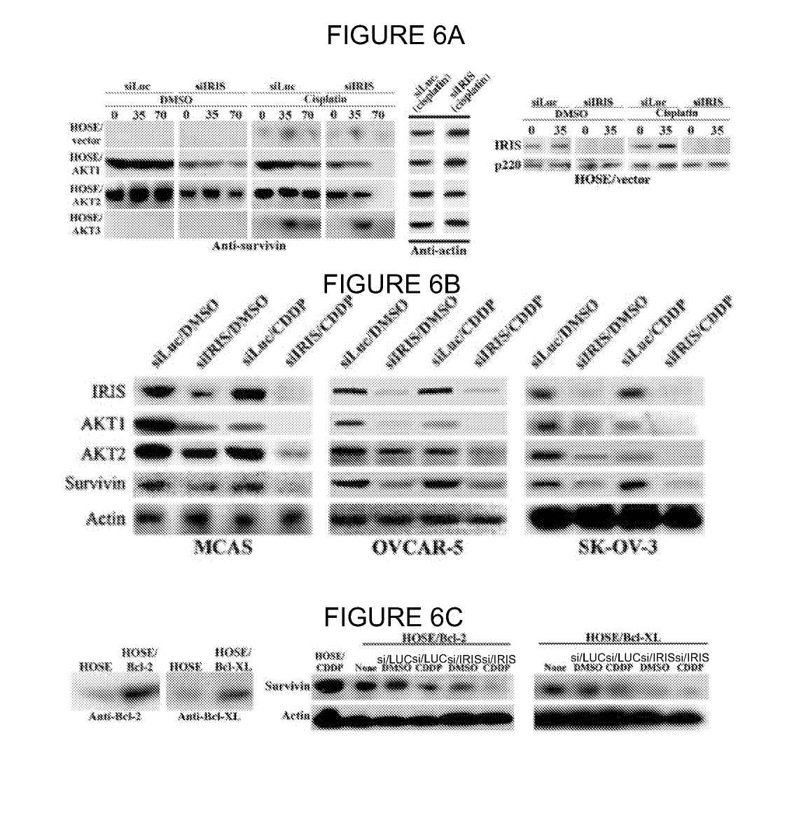

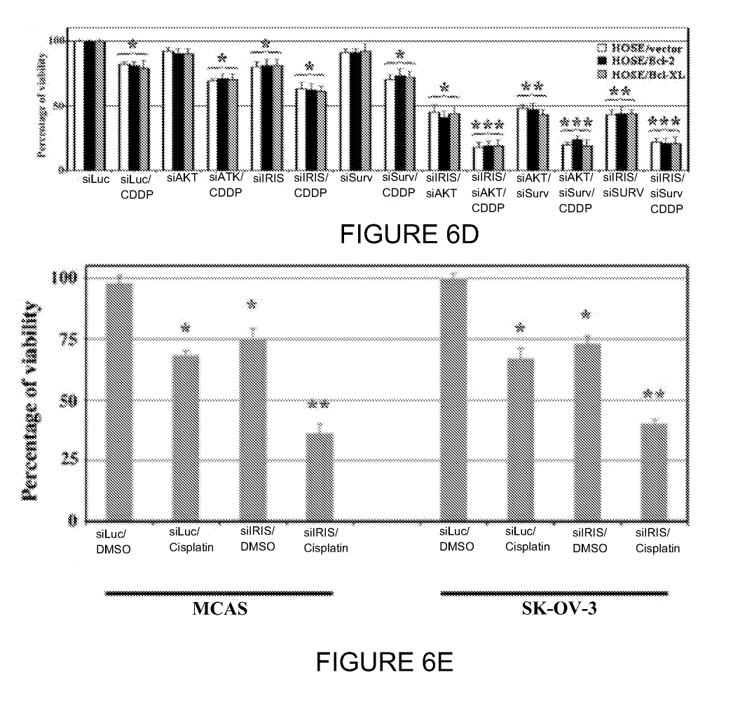

BRCA1-IRIS silencing sensitizes HOSE cells to cisplatin-induced survivin downregulation and cell death. If BRCA1-IRIS-AKT-survivin cascade were important to protect against cisplatin-induced cell death, one would expect that BRCA1-IRIS depletion would sensitize cells to cisplatin-induced loss of survivin expression and cell death. First, HOSE cell lines stably expressing two known survival factors Bcl-2 and Bcl-XL (42, FIG. 6C, left) were generated. HOSE/IRIS, /AKT1, /AKT2, /AKT3, /Bcl-2 or /Bcl-XL were transfected with control (luciferase or scrambled IRIS siRNA) or BRCA1-IRIS specific siRNA for 48 h before treatment with DMSO or cisplatin (35 or 70 μM) for an additional 24 h.

BRCA1-IRIS silencing had no effect on the expression of BRCA1/p220 in HOSE/vector or actin expression in HOSE/AKT1, /AKT2 or /AKT3 cell lines (FIG. 6A, right and middle). Treatment of HOSE/vector cells with 35 μM of cisplatin induced BRCA1-IRIS expression, which was effectively blocked by BRCA1-IRIS siRNA but had no effect on BRCA1/p220 expression (FIG. 6A, right). BRCA1-IRIS silencing reduced survivin expression regardless of AKT1, AKT2 or AKT3 overexpression (FIG. 6A, left). Treatment with 35 μM cisplatin induced survivin expression in vector and AKT3 expressing cells (FIG. 6A, left), but had no such effect in AKT1 and AKT2 expressing cells (FIG. 6A, left). Pre-silencing of BRCA1-IRIS further reduced the level of survivin in all cell lines (FIG. 6A, left). Moreover, these effects were even more pronounced when cells were treated with 70 μM of cisplatin (FIG. 6A, left).

Furthermore, HOSE/Bcl-2 or HOXE/Bcl-XL cell lines were treated in the same manner except that the effect of 35 μM cisplatin only was tested. Overexpression of Bcl-2 or Bcl-XL did not protect survivin against BRCA1-IRIS loss-, cisplatin- or both-induced downregulation (FIG. 6C, right). Moreover, equal number of HOSE/vector, /Bcl-2 or /Bcl-XL cells were transfected with AKT, survivin or BRCA1-IRIS siRNAs separately or in combination and then were treated or not with 35 μM of cisplatin followed by measurement of cell viability using MTT assay. Silencing of single molecule had little effect on HOSE/vector, /Bcl-2 or /Bcl-XL cells viability (FIG. 6D), whereas treatment of single molecule silenced cells with cisplatin significantly reduced HOSE cell viability by ˜25% (FIG. 6D). The combination of each two siRNAs reduced the viability by ˜50% (FIG. 6D), providing evidence that each molecule control overlapping as well as distinct cell survival and/or proliferation pathways. Adding low concentration of cisplatin, 35 μM to any of the siRNA combinations decreased cell viability even further to ˜75% (FIG. 6D), providing evidence that reducing BRCA1-IRIS, AKT or survivin expression (or activity) kills more cells in combination with low cisplatin concentration.

BRCA1-IRIS downregulation sensitizes ovarian cancer cell lines to cisplatin-induced survivin downregulation and cell death. In addition to the intrinsically high levels of BRCA1-IRIS in ovarian cancer cells, their stress-induced up-regulation of AKT and/or survivin in part through up-regulation of BRCA1-IRIS likely enhances the resistance to cisplatin or other anti-cancer agents. To test this hypothesis, ovarian cancer cell lines MCAS, OVCAR-5 and SK-OV-3 were transfected with control or BRCA1-IRIS siRNAs for 72 h and were exposed to DMSO or 35 μM of cisplatin during the last 24 h. As expected BRCA1-IRIS silencing downregulated the expression of AKT1, AKT2 and survivin to various degrees in all cell lines (FIG. 6B). Cisplatin (35 μM) alone modestly decreased the expression of these proteins (FIG. 6B), while the combination abolished the expression of all proteins in all cell lines (FIG. 6B).

Equal numbers of MCAS and SK-OV-3 cells were transfected with control or BRCA1-IRIS siRNAs for 72 h and exposed to DMSO or 35 μM of cisplatin during the last 24 h followed by MTT assay to measure cell viability. BRCA1-IRIS silencing or treatment with 35 μM of cisplatin decreased cell viability by 25-35% in both cell lines (FIG. 6D), the combination decreased it by >65% (FIG. 6D), providing evidence that reducing BRCA1-IRIS levels sensitizes ovarian cancer cells to low doses of cisplatin.

As previously noted, a cell's ability to evade cell death and to proliferate post geno-/cell-toxic stresses likely leads to formation of cancer. Activation of p38MAPK and p53 following these stresses helps protect cells against cancer development by initiating apoptosis. The duration of p38MAPK and p53 activation is regulated by the WIP1 phosphatase. BRCA1-IRIS triggers WIP1 expression in a p53-dependent and -independent manner. BRCA1-IRIS triggers the expression and cytoplasmic localization of the mRNA stabilization and translation inducer, HuR, that binds p53 and PPM1D mRNA. Hence, BRCA1-IRIS overexpression inactivates p38MAPK and/or p53 by upregulating WIP1 expression. BRCA1-IRIS abrogation of the homeostatic balance maintained by the p38MAPK-p53-WIP1 pathway suppressed cell death induced by a lethal dose of short-wavelength UV light, and high dosage of etoposide or H₂0₂, and allowed cells to survive and proliferate post geno-/cell-toxic stresses. This mechanism represents a new link between geno-/cell-toxic stress and aggressive breast cancer formation in p53 wild-type cells.

The tumor suppressor p53 is activated in response to a variety of mitogenic and stressful stimuli (Moll and Schramm, 1998; Hammond and Giaccia, 2005; Latonen and Laiho, 2005). Following genotoxic stresses, p53 expression increases primarily through modulation of the steady-state level of the protein through phosphorylation and acetylation (Shieh et al., 1997; Dumaz and Meek, 1999; Sykes et al., 2009). Stabilized p53 induces transcription of many cell cycle arrest and apoptosis genes, such as Gadd45 and p21WAF1, Bcl-2, Bax, PUMA and MDM2 (Moll and Schramm, 1998; Hammond and Giaccia, 2005; Latonen and Laiho, 2005). Moreover, the stabilizing factor, HuR, binds and stabilizes p53 mRNA following ultraviolet irradiation (Yao et al., 1993; Mazan-Mamczarz et al., 2003; Tong and Pelling, 2009). HuR is a nucleocytoplasmic shuttling protein that binds to the AU-rich element in the 3′ untranslated region of mRNAs of many genes, such as VEGF, p21, Cyclin A and B1, c-fos and p27 (Al-Mohanna et al., 2007; Sakuma et al., 2008).

Protein phosphatase magnesium-dependent 1 delta (PPM1D), the gene encoding for the serine/threonine phosphatase WIP1 (for wild-type p53-induced phosphatase 1), is located on human chromosome 17q23-24 (Fiscella et al., 1997). PPM1D amplification and overexpression is associated with poor clinical outcomes in neuroblastoma, breast and ovarian clear cell carcinomas (Hirasawa et al., 2003; Saito-Ohara et al., 2003; Bulavin et al., 2004). WIP1 is expressed under various stress conditions, such as infra-red and ultraviolet, in a p53-dependent manner, where it inactivates p38MAPK and p53 itself (Takekawa et al., 2000; Yamaguchi et al., 2005).

p38MAPK is a serine/threonine kinase, and is a member of the mitogen-activated protein kinase (MAPK) family that includes the extracellular signal-regulated protein kinases and the stress-activated c-Jun N-terminal kinases (Sturgill, 2008). Following geno-/cell-toxic stresses, p38MAPK is activated by phosphorylation of threonine 180/tyrosine 182 (TI80/Y182) (Fornace, 1999; Sturgill, 2008). Activated p38MAPK then activates p53 by phosphorylating its serine 15, 33 and 46, thereby inducing cell cycle arrest or apoptosis (Fornace, 1999).

Despite its original discovery as a product of the tumor suppressor BRCA1 locus, and despite sharing 1365 from its 1399 residues with the tumor suppressor protein product of this locus, BRCA1/p220, BRCA1-IRIS has oncogenic properties. BRCA1-IRIS promotes DNA replication during the S phase, in part by suppressing the inhibitory function of the DNA replication suppressor, Geminin (ElShamy and Livingston, 2004), and cell proliferation by upregulating the Cyclin D1 expression, directly by binding to its promoter through c-Jun/AP1 transcription complex and activates its transcription (Nakuci et al., 2006), or indirectly by suppressing the expression of the c-Jun N-terminal kinase-specific inactivating phosphatase DUSP3/VHR (Hao and ElShamy, 2007).

The following experiments and studies examine the mechanisms governing the production of breast cancer in wild-type p53 cells. These experiments were reported in an article entitled, “BRCA1-IRIS overexpression abrogates UV-induced p38MAPK/p53 and promotes proliferation of damaged cells,” K Chock, J M S Allison, and W M ElShamy; Oncogen 23; 29(38) 5274-85 (Sep. 23, 2010), herein incorporated by reference.

Short-wavelength UV light (UVC), etoposide or H₂O₂ exposure triggered BRCA1-IRIS expression in normal and breast cancer cell lines, which triggered WIP1 expression in a p53-dependent and -independent manner. Independently, BRCA1-IRIS promoted the expression and cytoplasmic localization of HuR that binds p53 and PPM1D mRNAs and enhanced their stability and translation. Accordingly, BRCA1-IRIS silencing induced apoptosis in breast cancer cells exposed to UVC, etoposide or H₂O₂, and p53 silencing or p38MAPK inactivation augmented that apoptosis. However, BRCA1-IRIS overexpression in normal human mammary epithelial (HME) cells suppressed cell death induced by UVC, etoposide or H₂O₂, and these damaged cells continued to proliferate, especially when p53 was silenced or p38MAPK was inactivated in them. The data provided below provides evidence that this mechanism accounts, at least in part, for the initiation of breast cancer in patients with the wild-type p53 gene.

Results

BRCA1-IRIS Triggers WIP1 Expression in a p53-Dependent and -Independent Manner

Silencing of BRCA1-IRIS activates p38MAPK (Hao and ElShamy, 2007). To test whether alterations in WIP1 expression is responsible for that effect (Hickson et al., 2007), ET474, MCF7 and SKBR3 breast cancer cell lines endogenously overexpressing BRCA1-IRIS (FIG. 7A) were transfected with luciferase (hereafter control) or BRCA1-IRIS small interfering RNAs (siRNAs). BRCA1-IRIS silencing significantly reduced WIP1 mRNA (FIG. 7B) and protein (FIG. 7D) levels in all the cell lines, leading to a significant increase in p-p38MAPK with no effect on the total p38MAPK level (FIG. 7D). BRCA1-IRIS silencing also reduced p53 mRNA (FIG. 7B) and total and phosphorylated protein (detected using a mix of p-S15/37/46-p53 antibodies (hereafter p-p53), FIG. 7D levels, consistent with WIP1 being the p53 target.

Three HME cell lines, one carrying uninducible (IRIS1) and two carrying inducible (IRIS2 and IRIS3) His-tagged BRCA1-IRIS alleles, were generated. IRIS2 and IRIS3 express three- to four-fold more BRCA1-IRIS compared with control cells when induced with 2 μg/ml of doxycycline for 72 h (FIG. 7A). BRCA1-IRIS overexpression increased p53 and WIP1 mRNA (FIG. 7C) and protein (FIG. 7E) levels compared with control cells, and had no effect on the total p38MAPK protein level (FIG. 7E). BRCA1-IRIS overexpression increased p-p53 (detected using a mix of p-S 15/37/46-p53 antibodies) level perhaps by an oncogene-induced cellular stress-related mechanism (Lowe, 1999). To identify the exact site on p53 that is phosphorylated in BRCA1-IRIS overexpressing cells, HME and induced IRIS2 and IRIS3 total cell extracts were probed with anti-p-S15, S37 or S46-p53 antibodies, separately. BRCA1-IRIS overexpression triggered phosphorylation of all three sites on p53 (FIG. 7A). No p-p38MAPK was observed in any of the cell lines (FIG. 7E), for which an explanation is given in the discussion.

BRCA1-IRIS silencing reduced WIP1 expression in p53 wild-type, MCF-7 and p53 mutant, BT474 and SKBR3 cells. The following study was conducted to determine whether BRCA1-IRIS induces WIP1 in a p53-independent manner. BRCA1-IRIS was silenced in MCF7 cells stably expressing p53 small interfering hairpin. Compared with control MCF7 cells, these cells expressed equal but ˜30% reduced BRCA1-IRIS and WIP1, respectively (FIG. 7F). BRCA1-IRIS silencing decreased WIP1 levels in both cell lines (FIG. 7F). BRCA1-IRIS silencing in human embryonic kidney cells infected with SV40 virus (HEK293T), in which SV40 large T antigen binds and inactivates p53 (based on low p21 expression, data not shown) also suppressed WIP1 expression (FIG. 7F). Finally, when the p53-negative osteosarcoma cell line SAOS2 was infected with His-tagged BRCA1-IRIS expressing vector an increase in WIP1 level compared with vector expressing cells was observed. See FIG. 7, showing p53-independent induction of WIP1 by BRCA1-IRIS.

BRCA1-IRIS Triggers p53 and WIP1 Expression in a Transcriptional-Independent Manner

Whether BRCA1-IRIS binds and activates p53 and PPM1D promoters was studied next. Human Cyclin D1 (positive control, see Nakuci et al., 2006), p53 and PPM1D promoter regions were cloned upstream of the luciferase gene in the pGL3-Basic vector to produce pGL3-hCycD1 (a 2.5 kb gene core promoter), -hp53 (a 140 by (−128−+12) gene core promoter) and -hPPM1D (a 849 bp gene core promoter) plasmids (see Materials and methods). According to (Wang and El-Deiry, 2006) p53 activates its own promoter, and (Lowe et al., 2010) nuclear factor kappa-light-chain-enhancer of activated B cells (NF-κB) activates the PPM1D promoter. In co-transfection experiments pcDNA3-p53 and pcDNA3-NF-κB/p65 triggered a >15-fold increase in pGL3-hp53 and pGL3-hPPM1D activity, respectively (FIG. 7G). Co-transfection of pcDNA3-BRCA1-IRIS did not activate transcription from pGL3-hp53 or pGL3-hPPM1D, even though it triggered a >20-fold increase in pGL3-hCycD1 activity (FIG. 7G). BRCA1-IRIS silencing (siGFP was used as negative control) in HME cells did not affect the activity of any of the promoters (FIG. 7G).

BRCA1-IRIS Enhances HuR Expression and Cytoplasmic Localization

Whether BRCA1-IRIS triggers p53 and WIP1 expression by a post-transcriptional mechanism was studied next. Whole cell lysates, nuclear (soluble and chromatin-bound), polysomal (cytoplasmic and ribosomal-bound) proteins were isolated from BRCA1-IRIS-silenced MCF7 and SKBR3 (after 48 h), or HME and induced IRIS2 and IRIS3 cell lines (for 72 h). Compared with control, BRCA1-IRIS silencing in MCF7 and SKBR3 cells reduced the total HuR levels (FIG. 8A), did not change nuclear HuR levels (FIG. 8B, left), but significantly reduced cytoplasmic (FIG. 8B, right) and polysomal HuR (FIG. 8B, upper panels) levels. This was also evident in immunofluorescence analysis (FIG. 8C). In contrast, compared with HME, induced IRIS2 and IRIS3 contained high total HuR levels (FIG. 8E), similar nuclear HuR levels (FIG. 8F, left), but higher cytoplasmic (FIG. 2F, right) and polysomal HuR (FIG. 8H, upper panels) levels. This was also evident in immunofluorescence analysis (FIG. 8G), providing evidence that BRCA1-IRIS stabilizes HuR in the cytoplasm rather than its cytoplasmic translocation.

HuR transcription is NF-κB controlled (Kang et al., 2008). Compared with control, BRCA1-IRIS silencing reduced total NF-κB/p65 in MCF7 and SKBR3 cells (FIG. 8A), whereas BRCA1-IRIS overexpression did not change total NF-κB/p65 (FIG. 8E). BRCA1-IRIS silencing did not affect the level of nuclear NF-κB/p65 in MCF7 cells but decreased it in SKBR3 cells (FIG. 8B). It increased the cytoplasmic NF-κB/p65 levels in MCF7 cells and decreased it slightly in SKBR3 cells (FIG. 8B). On the other hand, BRCA1-IRIS overexpression increased nuclear NF-κB/p65 level, while reduced it in the cytoplasm, (FIG. 8F) providing evidence that in BRCA1-IRIS-silenced MCF7 cells, retention of NF-κB/p65 in the cytoplasm, signal the cells to down-regulate the NF-κB/p65 expression, whereas in SKBR3 cells, NF-κB/p65 nuclear translocation is decreased. On the other hand, BRCA1-IRIS overexpression in HME cells triggers HuR expression by inducing NF-κB/p65 nuclear translocation. Regardless of the mechanism, the data collected provides evidence that BRCA1-IRIS enhances HuR expression in a NF-κB/p65-dependent manner.

BRCA1-IRIS Enhances HuR Binding to p53 and PPM1D mRNAs

By using RT-PCR analysis on HuR polysomal immunoprecipitation, compared with immunoglobulin G (negative control) immunoprecipitations that contained no detectable RNA in both cases (FIGS. 8D and 8H, lower panels), the polysomal HuR, immunoprecipitated from BRCA1-IRIS-silenced MCF-7 and SKBR3 cells contained lower levels of p53 and PPM1D mRNAs (FIG. 8D). HuR immunoprecipitated from induced IRIS3 contained higher levels of p53 and PPM1D mRNAs (FIG. 8H). There were no differences detected in nuclear (pre) p53 and PPM1D RNAs levels in BRCA1-IRIS-silenced MCF7 and SKBR3 or induced IRIS3 cells (FIGS. 8D and 8H). This provides evidence that BRCA1-IRIS triggers p53 and WIP1 expression by enhancing their mRNA stability and/or translation in the cytoplasm by HuR.

BRCA1-IRIS Silencing Triggers p38MAPK and p53 Activation by UVC

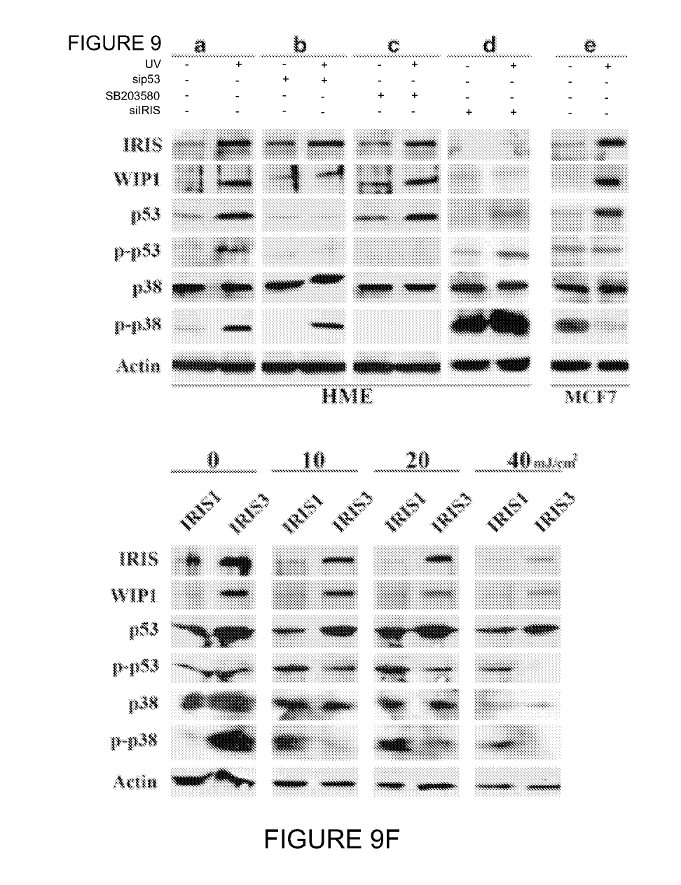

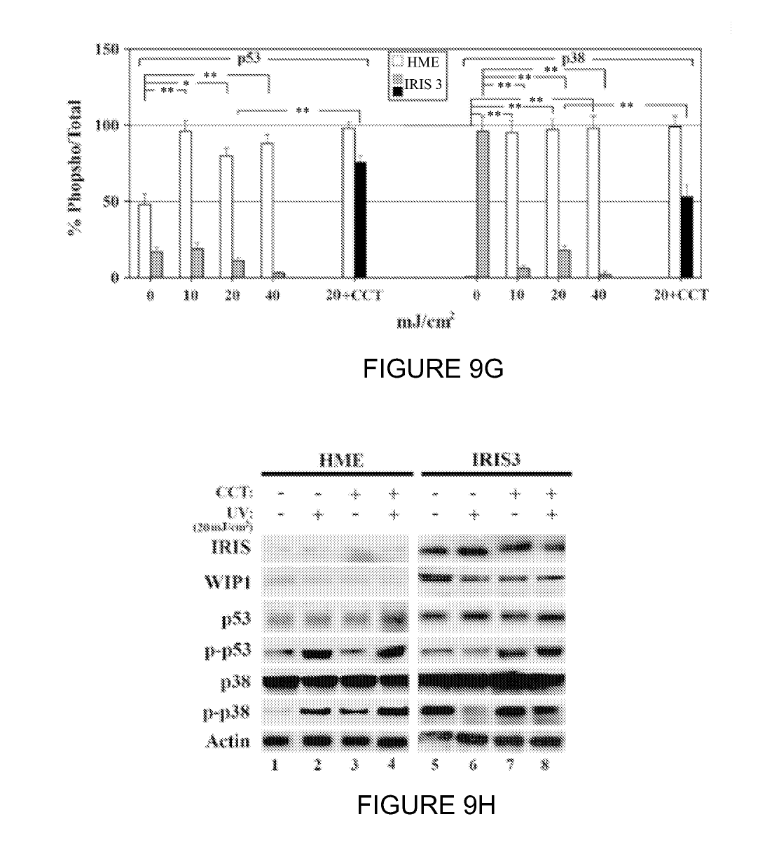

HME cells, with BRCA1-IRIS or p53 silenced (for 48 h), or treated with 10 μM of p38MAPK inhibitor, SB203580 (for 24 h) were exposed to a sub-lethal dose (20 mJ/cm²) of UVC or left untreated. In untreated cells, UVC induced BRCA1-IRIS, p53, p-p53 and WIP1 but not p38MAPK expression (FIG. 9A). Interestingly, UVC increased p-p38MAPK level in HME but decreased it in MCF7 cells (compare FIGS. 9A-9E). Without being tied to any specific theory, it is believed that the different manipulation of cells in every experiment differentially affects the p53 and p38MAPK phosphorylation. Indeed, untreated MCF7 cells, grown for 24 h in no-serum or in the presence of lipofectamine or the media was removed for 1 min and then added back and left to grow for 24 h, showed differences in p-p53 and p-p38MAPK (FIG. 7I).

In p53-silenced cells, UVC induced BRCA1-IRIS, WIP1 and p-p38MAPK expression, but had no effect on the total p38MAPK level (FIG. 9B), in keeping with the data in FIG. 7F, showing induction of WIP1 by a BRCA1-IRIS-dependent/p53-independent mechanism. In p38MAPK-inactivated cells, UVC induced BRCA1-IRIS, p53 and WIP1 but not p-p53 expression (FIG. 9C), in keeping with p53 as a p38MAPK target in UVC-exposed cells (see above). Finally, in BRCA1-IRIS-silenced cells, UVC exposure slightly increased p53 and p-p53 levels yet no increase in WIP1 level was observed (FIG. 9D), providing evidence that BRCA1-IRIS is critical for UVC-induced WIP1 expression and/or stability. Moreover, p-p38MAPK level was much higher in these cells compared with vehicle-treated cells even before UVC treatment (compare unexposed lanes in FIGS. 9A and 9D) increased even further following UVC exposure (FIG. 9D), perhaps due to geno-/cell-toxic stresses induced by BRCA1-IRIS silencing. This is further explained in the discussion.

BRCA1-IRIS Overexpression Abrogates UVC-Induced p38MAPK/p53 Activation in a WIP1-Dependent Manner

IRIS 1 and IRIS3 induced for 72 h were exposed to 0, 10, 20 or 40 mJ/cm² of UVC irradiation. After 24 h, compared with IRIS1, induced IRIS3 cells treated with 0 mJ/cm² UVC had higher levels of BRCA1-IRIS, p53, WIP1, p-p38MAPK, yet similar levels of p38MAPK and p-p53 (FIG. 9F). In 10 and 20 mJ/cm² treated cells, BRCA1-IRIS, p53 and WIP1 levels remained higher in induced IRIS3 and no change in p38MAPK level (FIG. 9F). While in IRIS1 cells p-p53 and p-p38MAPK levels rose significantly, in IRIS3 p-p53 and p-p38MAPK levels declined (FIG. 9F). Finally, in 40 mJ/cm² UVC-treated cells, BRCA1-IRIS, p53 and WIP1 levels were still detectable in induced IRIS3 but not in IRIS1 cells (FIG. 9F). In contrast, IRIS1 and not induced IRIS3 cells contained detectable levels of p-p53 and p-p38MAPK (FIG. 9F). The intensity of the p53, p-p53, p38MAPK and p-p38MAPK bands in FIG. 9F were measured using ImageJ program (NIH) to estimate the level of p53 and p38MAPK activation in BRCA1-IRIS overexpressing cells. About 50% of p53 in IRIS1 cells was phosphorylated before UVC exposure that rose to 80-100% after the exposure (FIG. 9G). In contrast, only 20% of p53 was phosphorylated in induced IRIS3 cells before UVC dropping to <10% after UVC (FIG. 9G). p38MAPK phosphorylation was undetectable in IRIS1 cells before UVC exposure but rose to ˜100% after UVC (FIG. 9G). In induced IRIS3 cells, the 100% of p38MAPK phosphorylated before UVC exposure dropped to <20% after UVC exposure (FIG. 9G).

To expand these data to other types of geno-/cell-toxic stresses, IRIS1 and induced IRIS2 (for 72 h) were treated with 0, 5, 10 or 50 μM of etoposide. After 24 h, IRIS1 cells showed low level of BRCA1-IRIS, p53 and WIP1 even after treatment with etoposide (FIG. 8I). Induced IRIS2 on the other hand maintained high level of BRCA1-IRIS, p53 and WIP1 even in the presence of 50 μM of etoposide and had similar level of total p38MAPK (FIG. 8I). Etoposide treatment triggered p53 phosphorylation in IRIS1 but not in the induced IRIS2 cells (Supplementary FIG. 8I). IRIS1 had low p-p38MAPK level before etoposide, increased significantly after etoposide treatment, whereas induced IRIS2 cells had high levels of p-p38MAKP before but sharply dropped after etoposide treatment. Taken together, these results suggest that BRCA1-IRIS overexpression prevents activation of p53 and/or p38MAPK by UVC and chemotherapeutic drugs.

WIP1 Suppression Restores p38MAPK/p53 Activation in BRCA1-IRIS Overexpressing Cells

CCT007093 is a novel WIP1 specific inhibitor. The HME and induced IRIS3 (72 h) cells were untreated or exposed to 20 mJ/cm² UVC, 10 μM of CCT007093 or both. BRCA1-IRIS, p53 and WIP1 level was low in HME and high in IRIS3 cells, whereas p38MAPK was high in both the cell lines. These levels were not changed in both cell lines following any of the treatments (FIG. 9H). p-p53 level was low in untreated HME and IRIS3 cells (FIG. 9H, lanes 1 and 5), rose after UVC treatment, decreased after CCT007093 treatment and increased in UVC+CCT007093-treated HME cells (FIG. 9H) Importantly, in induced IRIS3 cells, p-p53 level decreased after UVC treatment, increased after CCT007093 treatment and remained high after the combined treatment (FIG. 9H). Quantification of the ratios between p-p53 and p53 showed that in the presence of CCT007093, UVC induced phosphorylation of ˜75% of p53 in IRIS3 cells (FIG. 9G). p-p38MAPK was not detected in untreated HME cells (FIG. 9H, lanes 1 and 5), and was increased by UVC, CCT007093 and the combined treatment (FIG. 9H) Importantly, in induced IRIS3, the p-p38MAPK level was high before UVC, dropped after UVC and remained high in cells treated with CCT007093 alone or in combination with UVC (FIG. 9H). Quantification of the p-p38MAPK/p38MAPK ratio showed that in IRIS3 ˜55% of p38MAPK was phosphorylated by UVC in the presence of CCT007093 (FIG. 9G). These data are in keeping with that BRCA1-IRIS abrogation of p53 and p38MAPK activation after UVC treatment is WIP1-dependent.

Inactivating p53 or p38MAPK Protects Against Cell Death Induced by BRCA1-IRIS Silencing, UVC, Etoposide or H₂0₂ Treatment

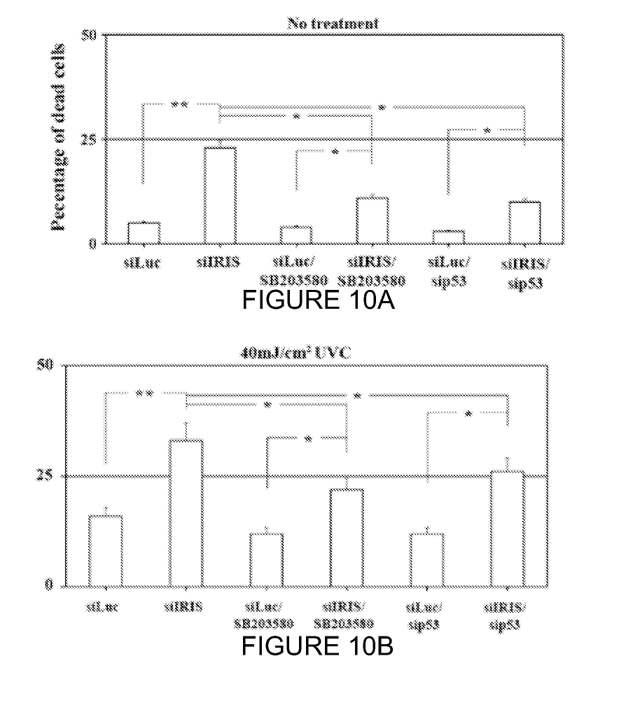

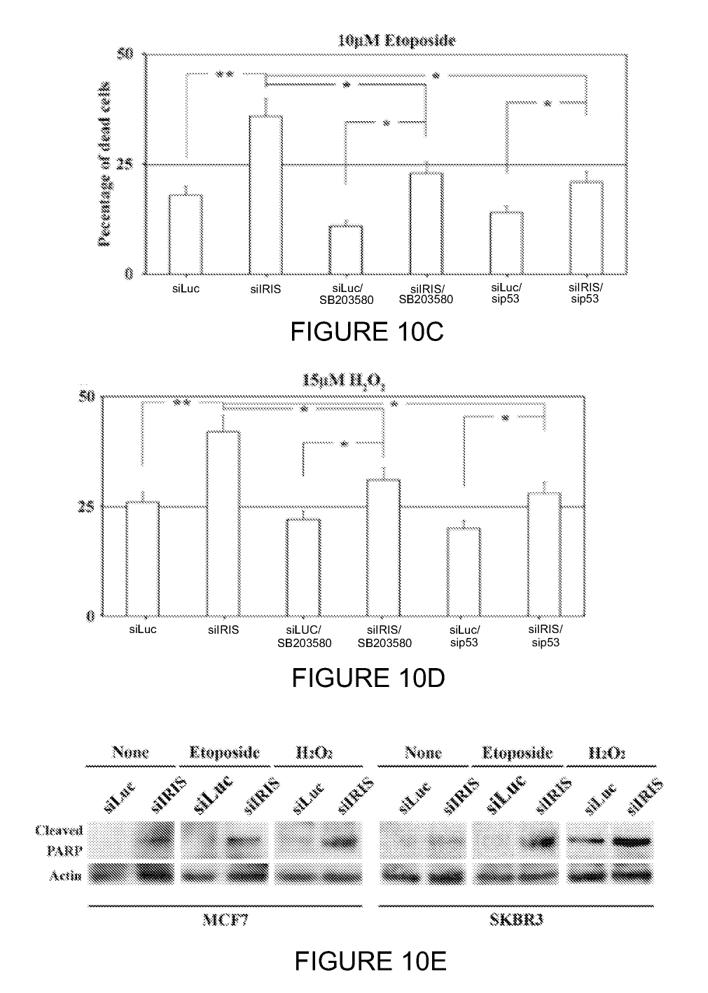

MCF7 and SKBR3 cell lines transfected with luciferase or BRCA1-IRIS siRNAs in the presence or absence of p38MAPK inhibitor (SB203580) or p53 siRNA were exposed to 40 mJ/cm² UVC, 10 μM etoposide or 15 μM H₂O₂. Using LIVE/DEAD assay (Invitrogen, Carlsbad, Calif., USA), we observed a significant increase in the number of dead cells 24 h after BRCA1-IRIS silencing in both the cell lines (FIG. 10A and FIG. 13). Inactivating p38MAPK or depleting p53 significantly reduced these effects (FIG. 10A and FIG. 13). This was confirmed by phase-contrast cell counting (FIG. 9I) and trypan blue exclusion using ViCell (Beckman Coulter, Fullerton, Calif., USA, FIG. 9J). UVC, etoposide or H₂O₂ treatment increased the level of cell death observed in all conditions, but the trend remained the same (FIGS. 10B, 10C and 10D, FIGS. 9I and 9J. Moreover, using the Apo-ONE Homogeneous Caspase-3/7 assay (Promega, Madison Wis., USA), there was an increase in active caspase3/7 levels in BRCA1-IRIS-silenced cells alone or in combination with UVC, etoposide or H₂O₂ treatment in both cell lines, and that p38MAPK inhibition or p53 silencing significantly reduces that level (FIG. 9K). Consistently, BRCA1-IRIS silencing alone or in combination with etoposide or H₂O₂ increased the level of cleaved poly-(ADP ribose) polymerase, the caspase-3 target during cell death (Nicholson and Thornberry, 1997), in both cell lines (FIG. 10E).

BRCA1-IRIS Protects Cells from UVC-, Etoposide- and H₂O₂-Induced Apoptosis by Suppressing p53 and p38MAPK Activities

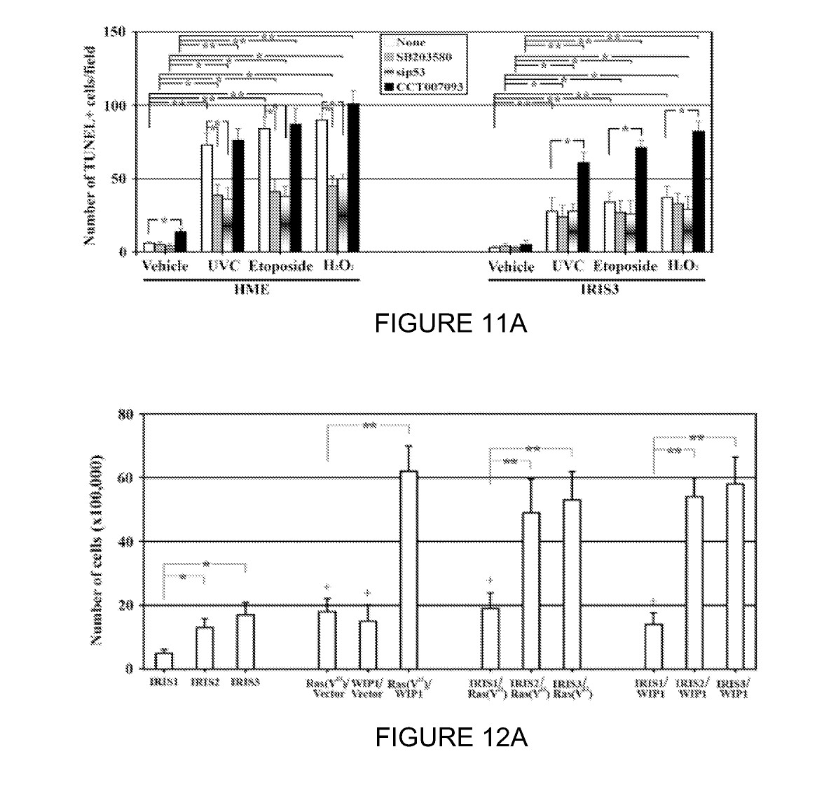

HME and induced IRIS3 (72 h) cells were exposed to 40 mJ/cm² UVC, 10 μM of etoposide or 15 μM of H₂O₂ in the presence or absence of 10 μM of SB203580, p53 siRNA or 10 μM of CCT007093. By using a Fluorescein FragEL DNA fragmentation detection kit to detect TUNEL⁺ cells, under no treatment conditions, CCT007093 increased slightly yet significantly the number of TUNEL⁺ cells in HME, but not in induced IRIS3 cells (FIG. 11A). Treatment with UVC, etoposide or H₂O₂ markedly increased the numbers of TUNEL⁺ cells in HME cells, while only modestly increasing the numbers in IRIS3 cells (FIG. 11 and FIGS. 10F, panels a-f). p38MAPK inhibition or p53 silencing significantly reduced the number of TUNEL⁺ cells under all treatment conditions in HME, but it stayed the same in induced IRIS3 cells (FIG. 11 and FIGS. 10F, panels g-r). Compared with vehicle control, CCT007093 treatment significantly increased the number of TUNEL⁺ cells in induced IRIS3 and not in HME cells (compare black with white bars in FIG. 11A and see FIG. 10F, panels s-x. This provides evidence that BRCA1-IRIS overexpression protects against geno-/cell-toxic stress-induced apoptosis by preventing p53 and p38MAPK activation in a WIP1-dependent manner.

BRCA1-IRIS Overexpression Induces Proliferation of UVC-Damaged Cells

Equal numbers of HME, IRIS2 or IRIS3 cells were grown in the presence of 2 μg/ml doxycycline and treated with 10 μM SB203580 or transfected with p53 siRNA or both before they were treated with 20 mJ/cm² UVC. At 24 h, MTT assay showed that BRCA1-IRIS overexpression alone increased cell viability after UVC treatment (compare bars 2 and 3 with 1 in FIG. 11B). Blocking UVC-induced apoptosis by silencing p53 or inactivating p38MAPK or both in HME cells increased the viability compared with UVC-untreated cells (compare bars 4, 7 and 10 with 1 in FIG. 11B). p53 silencing, p38MAPK inactivation or the combination increased even further the viability of IRIS2 and IRIS3 cells exposed to UVC (compare bars 5 and 6 to 4, bars 8 and 9 with 7 and 11 and 12 to 10 in FIG. 11B). This provides evidence that BRCA1-IRIS overexpression promotes proliferation of UVC-damaged cells by suppressing p53 and/or p38MAPK expression or activity in a WIP1-dependent manner.

BRCA1-IRIS Overexpression Transforms HME Cells with WIP1 or Oncogenic Ras

WIP1 cooperates with oncogenic Ras (Ras^{V12}) in transforming mammalian cells (Harrison et al., 2004). Whether BRCA1-IRIS also cooperates with WIP1 or Ras^{V12} in transforming HME cells was studied next. Empty (control), Ras^{V12} or WIP1-expressing vectors were transfected into IRIS 1, IRIS2 or IRIS3 cell lines and cells were induced and counted 72 h later, or were plated on soft agar plates, maintained in doxycycline and the colony size and number formed in each culture was measured 2-3 weeks later.

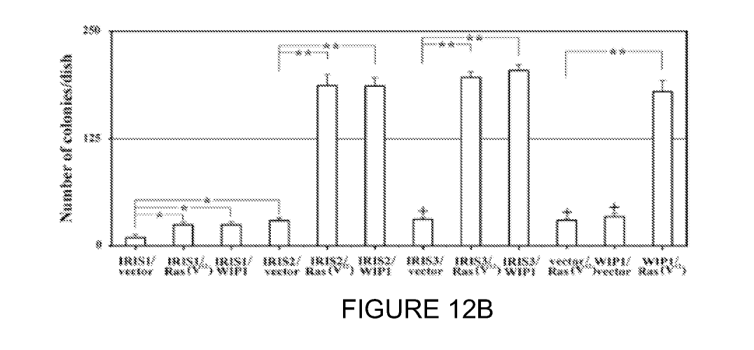

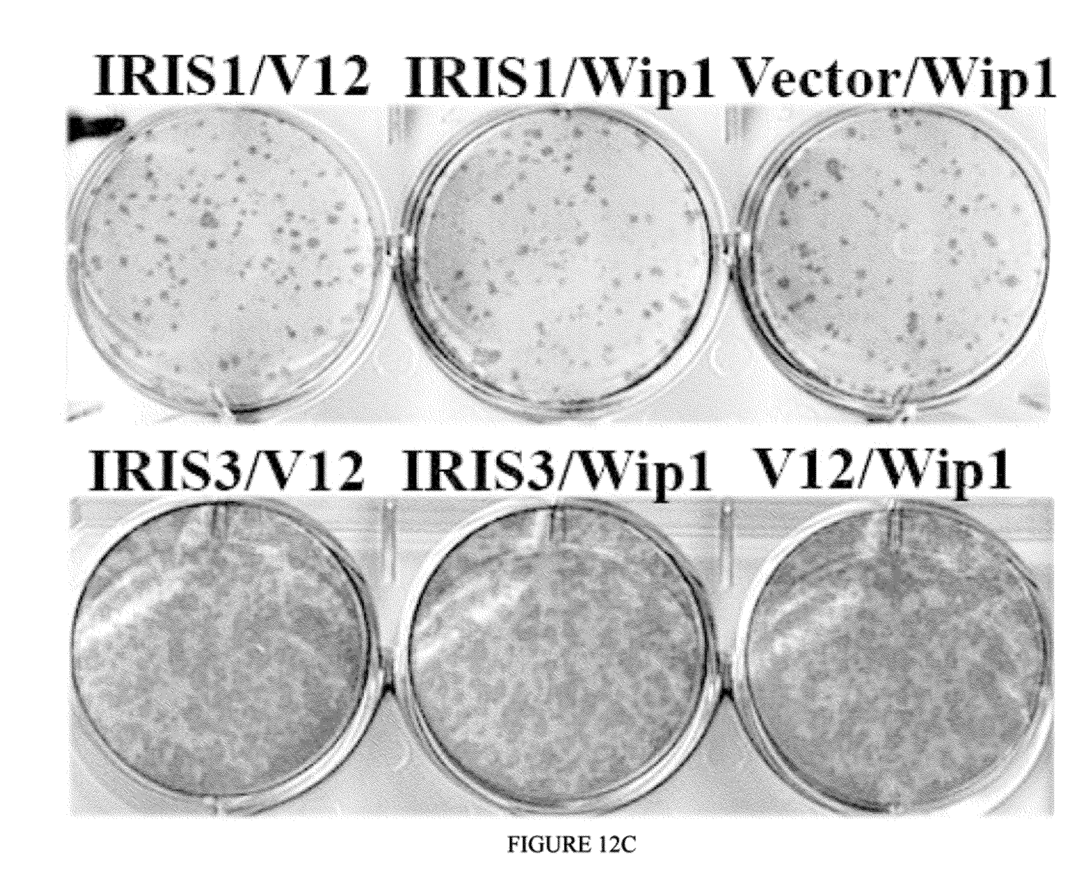

BRCA1-IRIS overexpression triggered two- to three-fold increase in cell number compared with control (FIG. 12A). Ras^{V12} and WIP1 overexpression (done in parental HME cells) each induced ·three-fold increase, while the combination led to >10-fold increase (FIG. 12A). Ras^{V12} or WIP1 overexpression in IRIS1 induced a modest two- to three-fold increase (FIG. 12A). Whereas Ras^{V12} or WIP1 overexpression in induced IRIS2 and IRIS3 cells led to eight- to ten-fold increase in cell number (FIG. 12A), suggesting that BRCA1-IRIS cooperates with Ras^{V12} or WIP1 in inducing HME cell proliferation.

In the soft agar experiments, while a low number of small-sized colonies was observed in the IRIS1 transfected with Ras^{V12} or WIP1 alone (FIG. 12B and FIG. 12C), transfection of either plasmid in induced IRIS2 or IRIS3 cells led to a greater number of larger-sized colonies (FIG. 12B and FIG. 12C), providing evidence that BRCA1-IRIS transforms HME cells with Ras^{V12} or WIP1, in vitro.

The data collected provides evidence that BRCA1-IRIS overexpression inactivates p38MAPK and p53 by inducing the expression of their WIP1 phosphatase, that BRCA1-IRIS triggering p53 and WIP1 expression is post-transcriptional, BRCA1-IRIS induces PPM1D and p53 mRNA stabilization and translation by promoting the expression and cytoplasmic availability of the mRNA-binding protein, HuR. This is in keeping with its ability to induce WIP1 expression in mutant as well as in wild-type p53 cells. Although HuR binding and stabilization of p53 mRNA has been reported earlier (AI-Mohanna et al., 2007; Tong and Pelling, 2009), the data above show post-transcriptional control of WIP1 expression by HuR.

HuR levels are high in gastric, breast, pancreatic, colon and oral tumors (Hasegawa et al., 2009; Licata et al., 2009; Lim et al., 2009; Marechal and Van Laethem, 2009), and in tumor cell lines no genetic or epigenetic alterations in the HuR gene have been found. Kang et al. (2008) recently showed that HuR overexpression depends on PI3′K/AKT signaling and NF-κB activity, and that AKT activation increased p65/RelA binding to a putative NF-κB-binding site in the HuR promoter. It was observed that BRCA1-IRIS overexpression induces AKT1 and AKT2 expression and stabilization in HME as well as ovarian surface epithelial cells (Chock et al., BRCA1-IRIS Overexpression Promotes Cisplatin Resistance in Ovarian Cancer Cells, Cancer Res; 70(21); 8782-91), herein incorporated by reference. Without being tied to any one theory or mechanism, the collected data presented here provides evidence that BRCA1-IRIS triggers HuR expression in a AKT/NF-κB-dependent manner and promotes aggressive breast and perhaps other cancers.

Activated p38MAPK induces checkpoints that arrest cells in G1/S or G2/M to allow time for repair pathways (Mikhailov et al., 2004; Reinhardt et al., 2007), in part by phosphorylating p53 (Huang et al., 1999; She et al., 2001) leading to the transcription of target genes such as Gadd45α, p21 and 14-3-3 (El-Deiry et al., 1993; Hermeking et al., 1997; Jin et al., 2003). Unlike cancer cells, BRCA1-IRIS silencing or overexpression activated the p38MAPK in HME cells. It is possible that in BRCA1-IRIS-silenced cells the induction of replication arrest (ElShamy and Livingston, 2004) activates p38MAPK, as was shown recently (Im and Lee, 2008; Rodriguez-Bravo et al., 2007). However, in undamaged BRCA1-IRIS overexpressing cells, perhaps p38MAPK is activated to induce cell survival and/or motility (Suarez-Cuervo et al., 2004; Demuth et al., 2007; Hsieh et al., 2007; Junttila et al., 2007). Alternatively, as p-p38MAPK phosphorylates HuR in the nucleus and enhances its cytoplasmic translocation (Gorospe, 2003; Tran et al., 2003; Lafarga et al., 2009), it is possible that in undamaged cells, this is how BRCA1-IRIS induces HuR cytoplasmic translocation. If true in vivo, then this could be a reason to develop an anti-IRIS-based therapy.

Materials and Methods

Cell Culture and UVC, Etoposide and H₂O₂ Treatments

BT474, MCF7 and SKBR3 cells were maintained in Roswell Park Memorial Institute medium (Invitrogen) supplemented with 10% fetal bovine serum and antibiotics. SOAS2 and HEK293T cells were maintained Dulbecco's Modified Eagle medium (Invitrogen) with 10% serum. HME maintenance was as described in ElShamy and Livingston, 2004. Cancer or HME cells were at 80% confluence in 10 cm² dishes and were treated with UVC (different doses), SB203580 (10 μM, Sigma, St. Louis, Mo., USA), etoposide (10 μM, Sigma) and H₂O₂ (15 μM, Sigma) or CCT007093 (10 μM, Santa Cruz, Santa Cruz, Calif., USA), and these drugs were added directly to the cells.

Antibodies

BRCA1-IRIS monoclonal antibody was produced. Sampler kits for p53 (#9919) and p38MAPK (#9913) were from Cell Signaling (Danvers, Mass., USA). p-p53 was detected individually or by using mixtures of several phospho-specific p53 (S15), (S37) and (S46) supplied in this kit. p-p38MAPK antibody was produced against p-T180/p-Y182 supplied in the kit. Mouse anti-HuR (#39-0600) and rabbit anti-cleaved poly-(ADP ribose) polymerase (#44698G) antibodies were from Invitrogen. Lamin BI (Oncogene, Boston, Mass., USA, #NAI2-100UG), NF-κB/p65 (Santa Cruz, C-20), WIP1 (Abgent, San Diego, Calif., USA, #AP84370) and Actin (Calbiochem, Gibbstown, N.J., USA, chem, #CPO1) were used.

Reporters Gene Construction

The p53 promoter report construct design was according to (Li et al., 2007). The resultant plasmids were named as pGL3-hp53. The PPM1D promoter report construct was designed according to standard protocol. The resultant plasmid was named as pGL3-hPPM1D. The pGL3-hCycD1 construct was described earlier (Nakuci et al., 2006). For construction of expression plasmids see Materials and methods Supplementary Information.

Transient Transfection

Cells were transiently transfected using Lipofectamine PLUS reagent (Invitrogen). The following protocol was used.

Reporters Gene Construction and Luciferase Transactivation Assay.

A fragment containing human p53 gene core promoter element was generated by amplifying a region from −128 to +12 of human genomic DNA (G304A, Promega) using 5′-gacaagcttcagTCGCTCGAGCAGGCGATTACTTGCCCTTACTT-3′ (SEQ ID NO:3) (forward) and 5′-tcgctcgagcagGCTCTAGACTTTTGAGAAGCTC-3′ (SEQ ID NO:4) (reverse) primers, respectively. The sequences in lowercase at the 5′-ends of the forward and reverse primers are recognition sites specifically designed for XhoI and HindIII restriction enzymes, respectively. After digestion with XhoI and HindIII and purification, the fragment was cloned into XhoI and HindIII sites upstream of the firefly luciferase reporter gene in the pGL3-Basic vector (Promega). The resultant plasmids were designated as pGL3-hp53. PPM1D reporter construct was designed by PCR from human genomic DNA (G304A, Promega) using the primers 5′-GGGCTCGAGTCCCCCTAGTAGCTGGAACTAC-3′ (SEQ ID NO:5) (forward) and 5′-GGGAAGCTTTAGGCGCTCGCCGGCCAACTA-3′ (SEQ ID NO:6) (reverse). The fragment was cloned into a TA-TOPO plasmid (Invitrogen). To construct the PPM1D reporter vector we inserted a 849-bp EcoRI to BamHI fragment digested from this 3 kb full length promoter and cloned it into EcoRI and BamHI sites upstream of the firefly luciferase reporter gene in the pGL3-Basic vector (Promega).

Luciferase assay was performed 24 hrs after transfection. Luciferase activity was determined using Dual-Glo Luciferase Assay System® (Promega) according to manufacturer's instructions. Fifty μl of Dual-Glo Luciferase reagent was added to the cells cultured in 100 μl of growth medium and cultures were incubated 10 min at RT prior to measure firefly luciferase activity on a Multiplate Reader Synergy HT (BIO-TEK). Fifty μl of the Dual-Glo Stop & Glo reagent was added subsequently in each well and the plate incubated for at least 10 min at RT before the renilla luciferase activity was read to allow measurements of firefly and renilla luciferase from a single sample. The activity of each promoter, expressed as arbitrary unit, was obtained directly by firefly/renilla luciferase ratio. All assays were performed a minimum of 3 times in triplicates.

Mammalian Expression Plasmids.

HA tagged BRCA1-IRIS, NFκB/p65 or p53 under CMV promoter in the pCDN3.1 were either obtained or generated in our lab using standard cloning techniques. A lentivirus construct for His-tagged BRCA1-IRIS was generated by PCR amplification of His-IRIS cDNA from the pRev-Tre-IRIS (ElShamy and Livingston, 2004) and cloned it in the plasmid pSMPUW-IRES-Blasticidin Lentiviral Expression Vector (Cell Biolabs). Virus was produced, concentrated and used to infect cells according to supplier instructions.

Stable Transfection

The plasmids pMKO.1-puro (control) or pMKO.1-shp53-puro were obtained from Addgene (Cambridge, Mass., USA). Retrovirus production and infection into MCF7 cells was done using standard protocols.

After infection, clones were made by puromycine selection. Ten clones were tested and the one that showed >95% knockdown was chosen to analyze further and was named MCF7/shp53. MCF7/vector expressed p53 level similar to uninfected cells.

For more details see Materials and methods in Supplementary Information.

Cell Fractionation

To prepare cytoplasmic, nuclear and polysomal fractions, the protocol of (Tenenbaum et al., 2002) was used.

For cytoplamic and nuclear proteins, cells were trypsinzed, washed and then agitated in 4° C. for 15 mins in buffer containing (110 mM KOAc, 15 mM NaAc, 2 mM MgOAc, 2 mM DTT, 20 mM Hepes pH 7.0 and 50 μg/ml Digitonin). Cells were then centrifuged at 1500 rpm for 15 mins at 4° C. Supernatant was taken as cytoplasmic fraction. Nuclear pellet was incubated in hypotonic buffer containing (1 mM Hepes pH 7.0, 0.5 mM EDTA pH 7.5, 0.5% NP-40) for 15 mins with agitation. After 10 mins of centrifugation at 1500 rpm at 4° C., supernatant was taken as nuclear fraction, while the pellet was resuspended in PBS and sonicated, and after centrifugation for 5 mins at 14000 rpm at 4° C., supernatant was taken as chromatin fraction.

Immunofluorescence

The protocol described in ElShamy and Livingston was used.

After treatment, cells were washed with PBS, fixed in PBS containing 4% paraformaldehyde at room temperature for 30 min, washed with PBS again, and incubated in PBS containing 0.1% Triton X-100 for 10 min After incubation in blocking buffer (PBS containing 0.1% Tween-20, 3% BSA and 3% goat serum) for 1 hr, the slides were incubated with mouse anti-HuR antibody in PBS containing 5% goat serum for 1 hr at 37° C., washed with PBS containing 0.1% Tween-20, and further incubated with fluorescence goat anti-mouse IgG (Invitrogen) for 30 mins in dark. After final wash with PBS, the slides were mounted in Vectashield with DAPI (Vector Laboratories) and visualized with an Evos fluorescence microscope (AIG).

RT-PCR Assays

Total RNA was isolated after treatments using TRIzol reagent (Invitrogen) and treated with a DNA-free kit (Ambion, Austin, Tex., USA) to eliminate genomic DNA contamination. SuperScript III One-step RT-PCR with Platinum Taq (Invitrogen).

BRCA1-IRIS:
(SEQ ID NO: 7)
forward 5′-GGTCTGAGTGACAAGGAATTGGTTTCAGATGATGA
AGAA-3′
and
BRCA1-IRIS
(SEQ ID NO: 8)
reverse 5′-TTAACTATACTTGGAAATTTGTAAAATGTG,
p53:
(SEQ ID NO: 9)
forward 5′-GGGGATATCTGTAACAGTTCCTGCATGGG-3′,
p53
(SEQ ID NO: 10)
reverse 5′-GGGGGATCCCTCTTCCTCTGTGCGCCG-3′,
PPM1D:
(SEQ ID NO: 11)
forward 5′-GCCAGAACTTCCCAAGGAAAG-3′
and
PPM1D
(SEQ ID NO: 12)
reverse 5′-GGTTCAGGTGACACCACAAATTC-3′,

GAPDH was used according to published data.

Immunoprecipitation of mRNP-Protein Complex and RT-PCR

The protocol used by (Tenenbaum et al., 2002) was used and is herein incorporated by reference.

Fifty μl of protein G Agarose Beads (Pierce) were pre-coated with 50 μg of anti-HuR or 5 μg of mouse IgG (Jackson ImmunoResearch Laboratories, Inc.) in 1 ml PBS total overnight at 4° C. with constant rocking. Next day, wash antibody coated beads 4 times with 1 ml cold NT2 Buffer (50 mM Tris pH 7.4, 150 mM NaCl, 1 mM MgCl₂, 0.05% Nonidet p-40) and centrifuge at 10000 rpm at 4° C. for 30 secs. Resuspend beads in 1 ml final volume of NT2 Buffer supplemented with 140 μl RNase A (Invitrogen), 5 μl 10 mg/ml Vanadyl ribonucleoside complexes, 1 μl of 1 M DTT, 40 μl of 0.5 M EDTA (Fisher) and 1 mg of polysomal lysate. Incubate overnight 4° C. with constant rocking. Wash beads 3 times with 1 ml cold PBS and centrifugate at 14000 rpm for 30 secs at 4° C. Resuspend beads in 47 μl PBS+3 μl of 10 mg/ml Proteinase K (Sigma) and incubate at 55° C. for 30 mins. Isolate RNA using the following protocol. Add 0.2 ml Phenol/Chloroform/Isoamyl alcohol pH 5.2 (Fisher Scientific), shake vigorously by hand for 15 secs, and incubate at room temperature (RT) for 3 mins Centrifuge at 4° C., followed at 12000 g for 15 mins Transfer the uppermost layer to a new tube and add 0.5 ml isopropyl alcohol. Incubate at RT for 10 mins followed by centrifugation at 4° C. at 12000 g for 10 mins Remove supernatant, add 1 ml 70% ethanol to RNAs and invert several times. Centrifuge at 7500 g at 4° C. for 5 mins Remove supernatant and air-dry the pelleted RNAs for 5-10 mins at RT. Resuspend the RNAs in 20 μl of RNAse free water. P53 and PPM1D primers used above were used in RT/PCR experiments. PCR products were visualized by electrophoresis in an ethidium bromide-stained 3% agarose gel.

RNA Interference Experiment

A dAdA-N19 double-stranded siRNA BRCA1-IRIS oligomer was synthesized and corresponded to a specific segment of the intron-11 sequence. A double-stranded 19-nucleotide luciferase- or green fluorescent protein-specific siRNAs was used as a negative control (Dharmacon, Lafayette, Colo., USA). Commercial p53 and WIP1 siRNA (Dharmacon) were used. The transfection of siRNA in breast cancer as well as HME cells was performed using Oligofectamine 2000 (Invitrogen) according to the manufacturer's instructions. Cells were harvested for 48 h or 72 h after transfection.

Cell Death by Fluorescence-Activated Cell Sorting Analysis

The LIVE/DEAD Fixable Dead Cell Stain Kit was used according to the supplier (Invitrogen) protocol.

TUNEL Detection Protocol

The Flurescein FragEL DNA Fragmentation Detection Kit was used according to the supplier (Calbiochem) protocol.

Active Caspase 3/7 Detection Protocol

The Apo-ONE Homogeneous Caspase-3/7 Kit was used according to the supplier (Promega) protocol.

MTS Assay

CellTiter 96 Aqueous Non-Radioactive Cell Proliferation Assay Kit (Promega) was used following the supplier instructions. As BRCA1-IRIS is a known inducer of replication and proliferation (ElShamy and Livingston, 2004; Nakuci et al., 2006; Hao and ElShamy, 2007), data are presented as (X treatment with UVC)/(X treatment without UVC)/(untreated HME with UVC)/(untreated HME without UVC), where X=given treatment.

Soft Agar Colony Formation Assay

Cells cultivated for 6 days with or without doxycycline (2 μg/ml) were plated in 0.3% agar (with or without doxycycline) on the plates, containing 0.5% agar (with or without doxycycline). The cells were cultivated on the plates for 2-3 weeks. Crystal violet stained colonies were counted under the microscope. Samples were assayed in triplicates.

Prepare 1% Noble Agar (Difco) and cool to 40° C. in a water bath. Warm 2× DMEM/F12+ additives to 40° C. in water bath. After 30 mins for temperature to equilibrate, mix equal volumes of the two solutions to give 0.5% Agar+1× DMEM/F12+ additives. In a 35 mm Petri dish, add 1.5 ml and allow to settle. Prepare 0.7% Agar in microwave and warm 2× DMEM/F12+ additives and bring both solutions to 40° C. in a water bath. Plate 5,000 cells/plate in 3 ml and incubate at 37° C. in humidified incubator for 10-14 days. Stain plates with 0.5 ml of 0.005% Crystal Violet for >1 hour, count colonies using a dissecting microscope. HEK293T cells were used as positive control and IMR90 cells as negative control.

Statistical Analysis

Comparisons of treatment outcomes were tested for statistical differences using the Student's t-test for paired data. Statistical significance was assumed at a P-value of <0.05.

### REFERENCES

The present description has referred to the following 50 references which are herein incorporated by reference.

1. Eltabbakh G, Awtrey C. Current treatment for ovarian cancer. Expert Opin Pharmacother 2001; 2(1):109-24.

2. McKeage M. New-generation platinum drugs in the treatment of cisplatin-resistant cancers. Expert Opin Investig Drugs 2005; 14(8):1033-46.

3. Tewari K, Mehta R, Burger R, et al. Emerging drugs for ovarian cancer. Expert Opin Emerg Drugs 2005; 10(2):413-42.

4. Sherman S, Lippard S. Structural aspects of platinum anticancer drug interaction with DNA. Chem Rev 1987; 87:1153-7.

5. Hersey P, Zhang X. Overcoming resistance of cancer cells to apoptosis. J Cell Physiol 2003; 196:9-18.

6. Coukos G, Rubin S. Chemotherapy resistance in ovarian cancer: new molecular perspectives. Obstet Gynecol 1998; 91(5 Pt 1):783-792.

7. Schrenk D, Baus P, Ermel N, et al. Up-regulation of transporters of the MRP family by drugs and toxins. Toxicol Lett 2001; 120(1-3):51-7.

8. Weaver D, Crawford E, Warner K, et al. ABCCS, ERCC2, XPA and XRCC1 transcript abundance levels correlate with cisplatin chemoresistance in non-small cell lung cancer cell lines. Mol Cancer 2005; 4(1):18.

9. Singer G, Stohr R, Cope L, et al. Patterns of p53 mutations separate ovarian serous borderline tumors and low-and high-grade carcinomas and provide support for a new model of ovarian carcinogenesis: a mutational analysis with immunohistochemical correlation. Am J Surg Pathol 2005; 29(2):218-24.

10. Lee S, Choi E, Jin C, et al. Activation of PI3K/Akt pathway by PTEN reduction and PIK3CA mRNA amplification contributes to cisplatin resistance in an ovarian cancer cell line. Gynecol Oncol 2005; 97(1):26-34.

11. Yang X, Zheng F, Xing H, et al. Resistance to chemotherapy-induced apoptosis via decreased caspase-3 activity and overexpression of antiapoptotic proteins in ovarian cancer. J Cancer Res Clin Oncol 2004; 130(7):423-8.

12. Deveraux Q, Reed J. IAP family proteins--suppressors of apoptosis. Genes Dev 1999; 13(3): 239-52.

13. Altieri D. Survivin in apoptosis control and cell cycle regulation in cancer. Prog Cell Cycle Res 2003; 5:447-52.

14. Tamm I, Wang Y, Sausville E, et al. IAP-family protein survivin inhibits caspase activity and apoptosis induced by Fas (CD95), Bax, caspases, and anticancer drugs. Cancer Res 1998; 58(23):5315-20.

15. Sui L, Dong Y, Ohno M, et al. Survivin expression and its correlation with cell proliferation and prognosis in epithelial ovarian tumors. Int J Oncol 2002; 21(2):315-20.

16. Takai N, Miyazaki T, Nishida M, et al. Expression of survivin is associated with malignant potential in epithelial ovarian carcinoma. Int J Mol Med 2002; 10(2):211-16.

17. Kawasaki H, Altieri D, Lu C, et al. Inhibition of apoptosis by survivin predicts shorter survival rates in colorectal cancer. Cancer Res 1998; 58:5071-74.

18. Monzo M, Rosell R, Felip E, et al. A novel anti-apoptosis gene: Re-expression of survivin messenger RNA as a prognosis marker in non-small-cell lung cancers. J Clin Oncol 1999; 17:2100-04.

19. Zhang M, Latham D, Delaney M, Chakravarti A. Survivin mediates resistance to anti-androgen therapy in prostate cancer. Oncogene 2005; 24(15):2474-82.

20. Nakamura M, Tsuji N, Asanuma K, et al. Survivin as a predictor of cis-diamminedichloroplatinum sensitivity in gastric cancer patients. Cancer Sci 2004; 95(1):44-51.

21. Tran J, Master Z, Yu J, et al. A role for survivin in chemoresistance of endothelial cells mediated by VEGF. Proc Natl Acad Sci USA 2002; 99(7):4349-54.

22. Papapetropoulos A, Fulton D, Mahboubi K, et al. Angiopoietin-1 inhibits endothelial cell apoptosis via the Akt/survivin pathway. J Biol Chem 2001; 275(13):9102-05.

23. Jiang K, Coppol D, Crespo N, et al. The phosphoinositide 3-OH kinase/AKT2 pathway as a critical target for farnesyltransferase inhibitor-induced apoptosis. Mol Cell Biol 2000; 20(1):139-48.

24. Fraser M, Leung B, Jahani-Asl A, et al. Chemoresistance in human ovarian cancer: the role of apoptotic regulators. Reprod Biol Endocrinol 2003a; 1:66.

25. Fraser M, Leung B, Yan X, et al. p53 is a determinant of X-linked inhibitor of apoptosis protein/Akt-mediated chemoresistance in human ovarian cancer cells. Cancer Res 2003b; 63(21):7081-88.

26. Bellacosa A, de Feo D, Godwin A, et al. Molecular alterations of the AKT2 oncogene in ovarian and breast carcinomas. Int J Cancer 1995; 64(4):280-5.

27. Cheng J, Godwin A, Bellacosa A, et al. AKT2, a putative oncogene encoding a member of a subfamily of protein-serine/threonine kinases, is amplified in human ovarian carcinomas. Proc Natl Acad Sci USA 1992; 89(19):9267-71.

28. Cheng J, Altomare D, Klein M, et al. Transforming activity and mitosis-related expression of the AKT2 oncogene: evidence suggesting a link between cell cycle regulation and oncogenesis. Oncogene 1997; 14(23):2793-801.

29. Clark A, West K, Streicher S, Dennis P Constitutive and inducible Akt activity promotes resistance to chemotherapy, trastuzumab, or tamoxifen in breast cancer cells. Mol Cancer Ther 2002; 1(9):707-7.

30. ElShamy W M, Livingston D M. Identification of BRCA1-IRIS, a BRCA1 locus product. Nat Cell Biol 2004; 6(10):954-67.

31. Furuta S, Jiang X, Gu B, et al. Depletion of BRCA1 impairs differentiation but enhances proliferation of mammary epithelial cells. Proc Natl Acad Sci USA 2005; 102(26):9176-81.

32. Wei M, Grushko T, Dignam J, et al. BRCA1 promoter methylation in sporadic breast cancer is associated with reduced BRCA1 copy number and chromosome 17 aneusomy. Cancer Res 2005; 65(23):10692-9.

33. Nakuci E, Mahner S, Direnzo J, ElShamy W M. BRCA1-IRIS regulates cyclin D1 expression in breast cancer cells. Exp Cell Res 2006; 312(16):3120-31.

34. Hao L, ElShamy W M. BRCA1-IRIS activates cyclin D1 expression in breast cancer cells by downregulating the JNK phosphatase DUSP3/VHR. Int J Cancer 2007; 121(1):39-46.

35. Tomlinson G, Chen T, Stastny V, et al. Characterization of a breast cancer cell line derived from a germ-line BRCA1 mutation carrier. Cancer Res 1998; 58(15):3237-42.

36. Yuan Y, Kim W, Han H, et al. Establishment and characterization of human ovarian carcinoma cell lines. Gynecol Oncol 1997; 66(3):378-87.

37. Kawakami H, Tomita M, Matsuda T, Ohta T, Tanaka Y, Fujii M, Hatano M, Tokuhisa T, Mori N. Transcriptional activation of survivin through the NF-kappaB pathway by human T-cell leukemia virus type I tax. Int J Cancer. 2005; 115(6):967-74.

38. Chock K, Allison J M, ElShamy W M. BRCA1-IRIS overexpression abrogates UV-induced p38MAPK/p53 and promotes proliferation of damaged cells. Oncogene. 2010 Jul. 12.

39. Guha M, Plescia J, Leav I, Li J, et al. Endogenous tumor suppression mediated by PTEN involves survivin gene silencing. Cancer Res. 2009; 69(12):4954-8.

40. Cross D, Alessi D, Cohen P, et al. Inhibition of glycogen synthase kinase-3 by insulin mediated by protein kinase B. Nature 1995; 378(6559):785-9.

41. Barrett R M, Osborne T P, Wheatley S P. Phosphorylation of survivin at threonine 34 inhibits its mitotic function and enhances its cytoprotective activity. Cell Cycle 2009; 8(2):278-83.

42. Ikeguchi M, Nakamura S, Kaibara N. Quantitative analysis of expression levels of bax, bcl-2, and survivin in cancer cells during cisplatin treatment. Oncol Rep 2002; 9(5):1121-26.

43. Di Maira G, Salvi M, Arrigoni G, et al. Protein kinase CK2 phosphorylates and upregulates Akt/PKB. Cell Death Differ 2005; 12(6):668-77.

44. Nascimento E B, Snel M, Guigas B, et al. Phosphorylation of PRAS40 on Thr246 by PKB/AKT facilitates efficient phosphorylation of Ser183 by mTORC1. Cell Signal 2010; 22(6):961-7.

45. Werzowa J, Cejka D, Fuereder T, et al. Suppression of mTOR complex 2-dependent AKT phosphorylation in melanoma cells by combined treatment with rapamycin and LY294002. Br J Dermatol 2009; 160(5):955-64.

46. Pallares J, Martinez-Guitarte J, Dolcet, X, et al. Survivin expression in endomaterial carcinoma: a tissue microarray study with correlation with PTEN and STAT3. Int J Gynecol Pathol. 2005; 24(3):247-53.

47. Nomura T, Yamasaki M, Nomura Y, et al. Expression of the inhibitors of apoptosis proteins in cisplatin-resistant prostate cancer cells. Oncol Rep 2005; 14(4):993-7.

48. Zaffaroni N, Daidone M. Survivin expression and resistance to anticancer treatments: perspectives for new therapeutic interventions. Drug Resist Updat 2002; 5(2):65-72.

49. Sommer K, Schamberger C, Schmidt G, et al. Inhibitor of apoptosis protein (IAP) survivin is upregulated by oncogenic c-H-Ras. Oncogene 2003; 22:4266-80.

50. Hoffman W, Biade S, Zilfou J, et al. Transcriptional repression of the anti-apoptotic survivin gene by wild-type p53. J Biol Chem 2002; 277:3247-57.

### ADDITIONAL REFERENCES

The following additional references cited in this description are also herein incorporated by reference.

Al-Mohanna M, Al-Khalaf H, Al-Yousef N, Aboussekhra A. (2007). The pl6INK4a tumor suppressor controls p21WAFl induction in response to ultraviolet light. Nucleic Acids Res 35: 223-233.

Bulavin D, Phillips C, Nannenga B, Timofeev O, Donehower L, Anderson C et al. (2004). Inactivation of the Wipl phosphatase inhibits mammary tumorigenesis through p38 MAPK-mediated activation of the p16(Ink4a)-p19(Arf) pathway. Nat Genet 36: 343-350.

Demuth T, Reavie L, Rennert J, Nakada M, Nakada S, Hoelzinger D et al. (2007). MAP-ing glioma invasion: mitogen-activated protein kinase kinase 3 and p38 drive glioma invasion and progression and predict patient survival. Mol Cancer Ther 6: 1212-1222.

Dumaz N, Meek D. (1999). Serine15 phosphorylation stimulates p53 transactivation but does not directly influence interaction with HDM2. EMBO J 18: 7002-7010.

ElShamy W M, Livingston D M. (2004). Identification of BRCA1-IRIS, a BRCA1 locus product. Nat Cell Biol 6: 954-967.

El-Deiry W, Tokino T, Velculescu V E, Levy D B, Parsons R, Trent J M et al. (1993). WAF1, a potential mediator of p53 tumor suppression. Cell 75: 817.

Fiscella M, Zhang H, Fan S, Sakaguchi K, Shen S, Mercer W et al. (1997). Wip1, a novel human protein phosphatase that is induced in response to ionizing radiation in a p53-dependent manner. Proc Natl Acad Sci USA 94: 6048-6053.

Fornace Jr A. (1999). Phosphorylation of human p53 by p38 kinase coordinates N-terminal phosphorylation and apoptosis in response to UV radiation. EMBO J 18: 6845-6854.

Gorospe M. (2003). HuR in the mammalian genotoxic response: post-transcriptional multitasking. Cell Cycle 2: 412-414.

Hammond E, Giaccia A. (2005). The role of p53 in hypoxia-induced apoptosis. Biochem Biophys Res Commun 331: 718-725.

Hao L, ElShamy W M. (2007). BRCA1-IRIS activates cyclin D1 expression in breast cancer cells by downregulating the JNK phosphatase DUSP3/VHR. Int J Cancer 121: 39-46.

Harrison M, Li J, Degenhardt Y, Hoey T, Powers S. (2004). Wip1-deficient mice are resistant to common cancer genes. Trends Mol Med 10: 359-361.

Hasegawa H, Kakuguchi W, Kuroshima T, Kitamura T, Tanaka S, Kitagawa Y et al. (2009). HuR is exported to the cytoplasm in oral cancer cells in a different manner from that of normal cells. Br J Cancer 100: 1943-1948.

Hermeking H, Lengauer C, Polyak K, He T C, Zhang L, Thiagalingam S et al. (1997). 14-3-3 sigma is a p53-regulated inhibitor of G2/M progression. Mol Cell 1: 3.

Hickson J A, Fong B, Watson P H, Watson A J. (2007). PP2Cdelta (Ppmld, WIP1), an endogenous inhibitor of p38 MAPK, is regulated along with Trp53 and Cdkn2a following p38 MAPK inhibition during mouse preimplantation development. Mol Reprod Dev 74: 821-834.

Hirasawa A, Saito-Ohara F, Inoue J, Aoki D, Susumu N, Yokoyama T et al. (2003). Association of 17q21-q24 gain in ovarian clear cell adenocarcinomas with poor prognosis and identification of PPM1D and APPBP2 as likely amplification targets. Clin Cancer Res 9: 1995-2004.

Hsieh Y, Wu T, Huang C, Hsieh Y, Hwang J, Liu J. (2007). p38 mitogen-activated protein kinase pathway is involved in protein kinase Calpha-regulated invasion in human hepatocellular carcinoma cells. Cancer Res 67: 4320-4327.

Huang C, Ma W Y, Maxiner A, Sun Y, Dong Z. (1999). p38 kinase mediates UV-induced phosphorylation of p53 protein at serine 389. J Biol Chem 274: 12229.

Im J S, Lee J K. (2008). A TR-dependent activation of p38 MAP kinase is responsible for apoptotic cell death in cells depleted of Cdc7. J Biol Chem 283: 25171-25177.

Jin S, Mazzacurati L, Zhu X, Tong T, Song Y, Shujuan S et al. (2003). Gadd45a contributes to p53 stabilization in response to DNA damage. Oncogene 22: 8536.

Junttila M, Ala-Aho R, Jokilehto T, Peltonen J, Kallajoki M, Grenman R et al. (2007). p38alpha and p38delta mitogen-activated protein kinase isoforms regulate invasion and growth of head and neck squamous carcinoma cells. Oncogene 26: 5267-5279.

Kang M, Ryu B, Lee M, Han J, Lee J, Ha T et al. (2008). NF-kappaB activates transcription of the RNA-binding factor HuR, via PI3K-AKT signaling, to promote gastric tumorigenesis. Gastroenterology 135: 2030-2042, 2042.e1-3.

Lafarga V, Cuadrado A, Lopez de Silanes I, Bengoechea R, Fernandez-Capetillo O, Nebreda A. (2009). p38 Mitogen-activated protein kinase- and HuR-dependent stabilization of p21(Cipl) mRNA mediates the G/S checkpoint. Mol Cell Biol 29: 4341-4351.

Latonen L, Laiho M. (2005). Cellular UV damage responses—functions of tumor suppressor p53. Biochim Biophys Acta 1755: 71-89.

Li I, Ke S, Ruiwen C, Yan H, Dan W, Shuhan S. (2007). p53 promoter-based reporter gene in vitro assays for quick assessment of agents with genotoxic potential. Acta Biochim Biophys Sin 39: 181-186.

Licata L, Hostetter C, Crismale J, Sheth A, Keen J. (2009). The RNA-binding protein HuR regulates GATA3 mRNA stability in human breast cancer cell lines. Breast Cancer Res Treat 122: 55-63.

Lim S, Lee S, Joo S, Song J, Choi S. (2009). Cytoplasmic Expression of HuR is Related to Cyclooxygenase-2 Expression in Colon Cancer. Cancer Res Treat 41: 87-92.

Lowe SW. (1999). Activation of p53 by oncogenes. Endocr Relat Cancer 6: 45-48.

Lowe J, Cha H, Yang Q, Fornace Jr A. (2010). Nuclear Factor-κB (NF-κB) Is a Novel Positive Transcriptional Regulator of the Oncogenic Wipl Phosphatase. J Bio Chem 285: 5249-5257.

Marechal R, Van Laethem J. (2009). HuR modulates gemcitabine efficacy: new perspectives in pancreatic cancer treatment. Expert Rev Anticancer Ther 9: 1439-1441.

Mazan-Mamczarz K, Galba'n S, Lo'pez de Silanes I, Martindale J, Atasoy A, Keene J et al. (2003). RNA-binding protein HuR enhances p53 translation in response to ultraviolet light irradiation. Proc Natl Acad Sci 100: 8354-8359.

Mikhailov A, Shinohara M, Rieder C. (2004). Topoisomerase II and histone deacetylase inhibitors delay the G2/M transition by triggering the p38 MAPK checkpoint pathway. J Cell Biol 166: 517.

Moll U, Schramm L (1998). p53—an acrobat in tumorigenesis. Crit Rev Oral Biol Med 9: 23-37.

Nakuci E, Mahner S, Direnzo J, ElShamy W M. (2006). BRCA1-IRIS regulates cyclin DI expression in breast cancer cells. Exp Cell Res 312: 3120-3131.

Nicholson D W, Thornberry N A. (1997). Caspases: killer proteases. TIBS 22: 299-306.

Reinhardt H, Aslanian A, Lees J, Yaffe M. (2007). p53-deficient cells rely on ATM- and ATR-mediated checkpoint signaling through the p38MAPK/MK2 pathway for survival after DNA damage. Cancer Cell 11: 175.

Rodriguez-Bravo V, Guaita-Esteruelas S, Salvador N, Bachs O, Agell N. (2007). Different S/M checkpoint responses of tumor and non tumor cell lines to DNA replication inhibition. Cancer Res 67: 11648-11656.

Saito-Ohara F, Imoto I, Inoue J, Hosoi H, Nakagawara A, Sugimoto T et al. (2003). PPM1D is a potential target for 17q gain in neuroblastoma. Cancer Res 63: 1876-1883.

Sakuma T, Nakagawa T, Ido K, Takeuchi H, Sato K, Kubota T. (2008). Expression of vascular endothelial growth factor-A and mRNA stability factor HuR in human meningiomas. J Neurooncol 88: 143-155.

She Q, Bode A M, Ma W Y, Chen N Y, Dong Z. (2001). Resveratrol-induced activation of p53 and apoptosis is mediated by extracellular-signal-regulated protein kinases and p38 kinase. Cancer Res 61: 1604.

Shieh S, Ikeda M, Taya Y, Prives C. (1997). DNA damage-induced phosphorylation of p53 alleviates inhibition by MDM2. Cell 91: 325-334.

Sturgill T. (2008). MAP kinase: it's been longer than fifteen minutes. Biochem Biophys Res Commun 371: 1-4.

Suarez-Cuervo C, Merrell M A, Watson L, Harris K W, Rosenthal E L, Väänänen HK et al. (2004). Breast cancer cells with inhibition of p38alpha have decreased MMP-9 activity and exhibit decreased bone metastasis in mice. Clin Exp Metastasis 21: 525.

Sykes S M, Stanek T J, Frank A, Murphy M E, McMahon S B. (2009). Acetylation of the DNA binding domain regulates transcription-independent apoptosis by p53. J Biol Chem 284: 20197-20205.

Takekawa M, Adachi M, Nakahata A, Nakayama I, Itoh F, Tsukuda H et al. (2000). p53-inducible Wip1 phosphatase mediates a negative feedback regulation of p38 MAPK-p53 signaling in response to UV radiation. EMBO J 19: 6517-6526.

Tenenbaum S A, Lager P, Carson C, Keene J. (2002). Ribonomics: identifying mRNA subsets in mRNP complexes using antibodies to RNA-binding proteins and genomic arrays. Methods 26: 191-198.

Tong X, Pelling J. (2009). Enhancement of p53 Expression in Keratinocytes by the Bioflavonoid Apigenin is Associated With RNA-Binding Protein HuR. Mol Care 48: 118-129.

Tran H, Maurer F, Nagamine Y. (2003). Stabilization of urokinase and urokinase receptor mRNAs by HuR is linked to its cytoplasmic accumulation induced by activated mitogen-activated protein kinase-activated protein kinase 2. Mol Cell Biol 23: 7177-7188.

Wang S, El-Deiry W S. (2006). p73 or p53 directly regulates human p53 transcription to maintain cell cycle checkpoints. Cancer Res 66: 6982-6989.

Yamaguchi H, Minopoli G, Demidov O, Chatterjee D, Anderson C, Durell S et al. (2005). Substrate specificity of the human protein phosphatase 2C delta, Wip1. Biochemistry 44: 5285-5294.

Yao K M, Samson M L, Reeves R, White K. (1993). Gene elav of Drosophila melanogaster: a prototype for neuronal-specific RNA binding protein gene family that is conserved in flies and humans. J Neurobiol 24: 723-739.

One of ordinary skill in the art will recognize that additional embodiments and implementations are also possible without departing from the teaching of the present invention or the scope of the exemplary claims which follow. This description and, particularly, the specific details of the exemplary implementation disclosed is given primarily for clarity of understanding and no unnecessary limitations are to be understood therefrom, for modifications will be apparent and obvious to one of ordinary skill in the art upon reading this disclosure and may be made without departing from the spirit or scope of the present invention.

What is claimed is:

1. A method for identifying risk for developing aggressive cancer in a patient, said method comprising:
selecting a patient to determine ones risk of developing aggressive cancer;
acquiring a biological sample from the patient;
determining the amount of BRCA1-IRIS expression present in the biological sample; and
identifying the patient as a risk candidate for developing aggressive cancer if the amount of BRCA1-IRIS expression exceeds a threshold amount of expression.

2. The method of claim 1, wherein the acquiring a biological sample comprises biopsying a tumor from the patient.

3. The method of claim 2, wherein determining the amount of BRCA1-IRIS expression comprises determining the amount of BRCA1-IRIS protein present in cells of the tumor.

4. The method of claim 1, wherein determining the amount of BRCA1-IRIS comprises determining the amount of BRCA1-IRIS protein present in tumor cells of the patient.

5. The method of claim 4, wherein determining the amount of BRCA1-IRIS comprises determining the amount of BRCA1-IRIS mRNA present in tumor cells of the patient.

6. A method for detecting expression of BRCA1-IRIS in a patient, the method comprising:
selecting a patient to determine the amount of BRCA1-IRIS expression;
acquiring a biological sample from the patient;
contacting the biological sample with a monoclonal antibody specific for an epitope of BRCA1-IRIS ; and
quantifying the amount of BRCA1-IRIS present in the sample based on an amount of the antibody bound BRCA1-IRIS.

7. The method of claim 6, wherein the monoclonal antibody recognizes a segment in intron 11 of BRCA1-IRIS.

8. The method of claim 6, further comprising identifying the patient as a risk candidate for developing aggressive cancer if the amount of BRCA1-IRIS exceeds a threshold amount of expression.

9. A method for detecting expression of BRCA1-IRIS in a patient, the method comprising:
selecting a patient to determine the amount of BRCA1-IRIS expression;
acquiring a biological sample from the patient;
extracting mRNA from the biological sample;
conducting PCR on the mRNA in the biological sample using specific 5′ and 3′ primers for BRCA1-IRIS; and
quantifying the amount of BRAC1-IRIS mRNA based on PCR product to determine the amount of BRAC1-IRIS expression.

10. The method of claim 9, wherein a forward primer has sequence SEQ ID No:1 and a reverse primer has sequence SEQ ID No:2.

11. The method of claim 9, further comprising identifying the patient as a risk candidate for developing aggressive cancer if the amount of BRCA1-IRIS expression exceeds a threshold amount of expression.

12. The method of claim 9, wherein the PCR product in amplification of around 200 base pairs of BRCA1-IRIS.

13. The method of claim 1, wherein the aggressive cancer is metastatic cancer.

14. The method of claim 8, wherein the aggressive cancer is metastatic cancer.

15. The method of claim 11, wherein the aggressive cancer is metastatic cancer.

16. A method for reducing the growth or proliferation of cancer cells, the method comprising administering a therapeutically effective amount of an inhibitor of BRCA1-IRIS, to cancer cells, to thereby reduce the growth or proliferation of the cancer cells.

17. The method of claim 16, wherein the inhibitor is siRNA which binds to BRCA1-IRIS mRNA, to thereby reduce the amount of BRCA1-IRIS protein produced by the cancer cells.

18. The method of claim 17, wherein the administered siRNA induces BRCA1-IRIS mRNA degradation when introduced to cancer cells expressing normal or high levels of BRCA1-IRIS mRNA.


SEQUENCE LISTING (12 sequences)

SEQ ID NO: 1 - DNA, 30 bp, Artificial Sequence
  synthesized primer sequence
ttcttccaaacaaatgaggcatcagtctga

SEQ ID NO: 2 - DNA, 40 bp, Artificial Sequence
  synthesized primer sequence
gttcctttaactatacttggaaatttgtaaaatgtgctcc

SEQ ID NO: 3 - DNA, 44 bp, Artificial Sequence
  synthesized primer sequence
gacaagcttcagtcgctcgagcaggcgattacttgcccttactt

SEQ ID NO: 4 - DNA, 34 bp, Artificial Sequence
  synthesized primer sequence
tcgctcgagcaggctctagacttttgagaagctc

SEQ ID NO: 5 - DNA, 31 bp, Artificial Sequence
  synthesized primer sequence
gggctcgagtccccctagtagctggaactac

SEQ ID NO: 6 - DNA, 30 bp, Artificial Sequence
  synthesize primer sequence
gggaagctttaggcgctcgccggccaacta

SEQ ID NO: 7 - DNA, 39 bp, Artificial Sequence
  synthesized primer sequence
ggtctgagtgacaaggaattggtttcagatgatgaagaa

SEQ ID NO: 8 - DNA, 30 bp, Artificial Sequence
  synthesized primer sequence
ttaactatacttggaaatttgtaaaatgtg

SEQ ID NO: 9 - DNA, 29 bp, Artificial Sequence
  synthesized primer sequence
ggggatatctgtaacagttcctgcatggg

SEQ ID NO: 10 - DNA, 27 bp, Artificial Sequence
  sythesized primer sequence
gggggatccctcttcctctgtgcgccg

SEQ ID NO: 11 - DNA, 21 bp, Artificial Sequence
  synthesized primer sequence
gccagaacttcccaaggaaag

SEQ ID NO: 12 - DNA, 23 bp, Artificial Sequence
  synthesized primer sequence
ggttcaggtgacaccacaaattc